(19) United States
(12) Patent Application Publication
(10) Pub. No.: US 2005/0084495 A1
(43) Pub. Date: Apr. 21, 2005

(54) Altering A B cell pathology using self-derived antigens in conjunction with specific-binding cytoreductive agent

(75) Inventor: Daniel P. Gold, Del Mar, CA (US)

Correspondence Address: WILSON SONSINI GOODRICH & ROSATI, 650 PAGE MILL ROAD, PALO ALTO, CA, 943041050, US

(73) Assignee: Favrille, Inc., San Diego, CA (US)

(21) Appl. No.: 10/948,894

(22) Filed: Sep. 23, 2004

Related U.S. Application Data
(60) Provisional application No. 60/560,305, filed on Apr. 6, 2004.
(60) Provisional application No. 60/505,497, filed on Sep. 23, 2003.

(51) Int. Cl. A61K 39/395

(52) U.S. Cl. 424/155.1

(57) ABSTRACT

A method of treating B cell malignancies or a method for preparing compositions for treating B cell malignancies wherein administration of a specific binding cytoreductive agent is followed by immunization with an autologous Id protein. The two treatments may be sequential, where the administration of the specific binding cytoreductive agent is completed before the administration of the autologous Id protein, or the administration of the specific binding cytoreductive agent and the immunization with an autologous Id protein may occur in an overlapping time course.

### RELATED APPLICATIONS

This application claims priority to the U.S. Provisional Application No. 60/560,305 entitled “Altering a B Cell Pathology Using Self-Derived Antigens in Conjunction With Specific-Binding Cytoreductive Agent” filed Apr. 6, 2004, and the U.S. Provisional Application No. 60/505,497 entitled “Altering a B Cell Pathology Using Self-Derived Antigens in Conjunction With Specific-Binding Cytoreductive Agent” filed Sep. 23, 2003.

### BACKGROUND OF THE INVENTION

1. Field of the Invention

This invention relates generally to the field of immunology and immunotherapy. More specifically, this invention relates to methods and compositions for altering B cell mediated pathologies, such as B cell malignancies and/or autoimmune diseases, by using a combination of therapies.

2. Background of the Invention

Cancers of the Circulatory System

Several types of cancers have their origin in the circulatory system. Among the major types are leukemias, neoplasms of the bone marrow and blood; myelomas, cancers of B cells; and lymphomas, cancers that originate in the lymphatic system. Lymphomas may be further subdivided into several groups; among the clinically most important lymphomas are non-Hodgkin's lymphomas (NHL).

Non-Hodgkin's lymphomas in turn form a diverse group of cancers. Three broad categories of these lymphomas are defined according to the International Working Formulation for tumor classification: low grade, intermediate grade and high grade. These differ in their aggressiveness and response to therapy (Cheson, et al., “Report of an International Workshop to Standardize Response Criteria for Non-Hodgkin's Lymphomas,” J. Clin. Oncol. 17(4):1244, 1999). Low-grade lymphoma usually presents as a nodal disease, and is often indolent or slow-growing. Intermediate- and high-grade disease usually present as much more aggressive cancers with large extranodal tumors. Overall, these lymphomas collectively rank fifth in the United States in terms of cancer incidence and mortality, and approximately 50,000 new cases are diagnosed each year.

Patients with NHL may also be classified using the Ann Arbor classification system: Stage I—involvement of a single lymph node region or localized involvement of a single extralymphatic organ or site. Stage II—involvement of two or more lymph node regions on the same side of the diaphragm or localized involvement of a single associated extralymphatic organ or site and its regional lymph nodes with or without other lymph node regions on the same side of the diaphragm. Stage III—involvement of lymph node regions on both sides of the diaphragm, possibly accompanying localized involvement of an extralymphatic organ or site, involvement of the spleen, or both. Stage IV—disseminated (multifocal) involvement of 1 or more extralymphatic sites with or without associated lymph node involvement or isolated extralymphatic organ involvement with distant (non-regional) nodal involvement. In Ann Arbor system, stages I, II, III, and IV of adult NHL are subdivided into A and B categories depending on whether the patient has well-defined generalized symptoms (B) or not (A). The B designation is associated with patients displaying the following symptoms: unexplained loss of more than 10% body weight in the 6 months prior to diagnosis, unexplained fever with temperatures above 38° C. and drenching night sweats. For further details, see The International Non-Hodgkin's Lymphoma Prognostic Factors Project: A predictive model for aggressive non-Hodgkin's lymphoma. New Eng. J. Med. 329(14): 987-994 (1993).

Treatments for Lymphomas and Leukemias

Such neoplastic lymphoproliferative diseases, including both leukemias and lymphomas, have been the focus of a great number pharmaceutical, surgical and immunological treatment regimens with varying degrees of success. In spite of significant progress in treating certain types, such as low grade, B cell non-Hodgkin's lymphoma in particular, the vast majority of patients relapse after responding to the currently best available treatment. Patients typically begin with cytoreductive chemotherapy using a combination of agents referred to by their acronyms (e.g., MOPP for mechlorethamine, vincristine (Oncovin®), prednisone, and procarbazine; CHOP for cyclophosphamide, doxorubicin, vincristine, and prednisone; CVP for cyclophosphamide, vincristine and prednisone; and EPOCH for etoposide, prednisone, vincristine, cyclophosphamide and doxorubicin). For relapsed patients, a new therapy was recently approved; now such patients are commonly treated with passive immunotherapy in the form of the anti-CD20 monoclonal antibody Rituxan® or similar antibodies. This therapy frequently results in a temporary remission.

Chemotherapy

Frequently, the first round of treatment in lymphomas, and leukemias is chemotherapy. A wide variety of chemotherapy approaches are available in the art to treat B cell and T cell malignancies. In general, combinations of chemotherapeutic agents are used as discussed supra such as CHOP, CVP and EPOCH. For a detailed discussion of the chemotherapeutic agents and their method of administration, see Dorr, et al., Cancer Chemotherapy Handbook, 2d edition, Appleton & Lange (Connecticut, 1994) hereby incorporated by reference. Further descriptions and protocols for chemotherapy regimes are well known to those skilled in the art.

Monoclonal Antibodies

Recent treatments have been developed in treating B cell lymphomas with antibodies directed against the CD20 antigen found on the surface of B cells. CD20 is an attractive target on B cell lymphomas due to its high levels of expression on the surface of malignant B cells. Further, the CD20 antigen is a logical target for therapy as (1) it is not shed by tumor B cells, (2) it is not internalized by tumor B cells, and (3) its expression is not modulated. CD20 in non-diseased tissue is an antigen expressed by B lymphocytes during early pre-B cell development and remains expressed on the surface until the B cell differentiates to a plasma cell. It is believed that the CD20 molecule regulates a step in the process of B cell activation that is required for cell cycle initiation and differentiation.

Anti-CD20 antibodies have been reported as therapies either alone or in combination with a second antibody or other chemotherapeutic agents. (See, e.g., U.S. Pat. No. 6,455,043 B1, issued on Sep. 24, 2002.) Rituxan®, an anti-CD20 antibody, has been approved by the Food and Drug Administration for use in relapsed and previously treated low-grade non-Hodgkin's lymphoma (NHL). Treatment with a related monoclonal antibody, Zevalin®, has also been approved (see, e.g., Witzig T E, et al., “Randomized controlled trial of yttrium-90-labeled ibritumomab tiuxetan radioimmunotherapy versus rituximab immunotherapy for patients with relapsed or refractory low-grade, follicular, or transformed B-cell non-Hodgkin's lymphoma,” J Clin Oncol. (2002) May 15;20(10):2453-63). A further anti-CD20 antibody, Bexxar® (tositumomab), has been recently approved by the FDA for the treatment of B cell lymphomas. Other monoclonal antibody therapies in clinical trials for the treatment of B cell lymphomas include Oncolym® (directed against the HLA-Dr10 protein), epratuzumab (directed against CD22), and an anti-CD52 antibody (Campath®/MabCampath®) (alemtuzumab).

Although their mechanism of action is still in dispute, anti-CD20 monoclonal antibodies are implicated in producing a therapeutic effect via the induction of apoptosis. Apoptosis allows the body to regulate cell number in a variety of processes including development, differentiation, and cell turnover. One characteristic change of cells undergoing apoptosis is an endonuclease activity associated degradation of DNA into a ladder-like appearance when resolved by gel electrophoresis. Other characteristic changes include membrane blebbing and other cytoskeletal disruptions, cell shrinkage and-loss of contact with neighboring cells, and, alterations in the plasma membrane. Various inducers of apoptosis have been identified including: TGF-β, TNF, Fas ligand, certain neurotransmitters, calcium, glucocorticoids, bacterial toxins, tumor suppressors, and chemotherapeutic agents such as cisplatin, doxorubicin, bleomycin, prednisone, etc. Additionally, apoptosis may be induced by physical damage such as heat shock, viral infection, free radicals, and gamma radiation; and toxins such as β-amyloid peptide.

Immunotherapy

In addition to the above methods of treatment, several experimental treatments for B cell cancers based on stimulating a patient's own immune system have been developed. Initial attempts at developing immunology-based treatments directed at antigens produced uniquely by malignant B cells involved laboriously isolating and purifying idiotypic (Id) proteins directly from the pathological B cells. This technique was first used in a mouse model system which used active immunization against idiotypic determinants on these isolated proteins to provide resistance to tumor growth (Daley et al., J. Immunol. 120(5):1620-24, 1978; Sakato et al., Microbiol. Immunol. 23(9):927-31, 1979). These results were confirmed in a number of additional experimental tumor models (Stevenson et al., J. Immunol. 130(2):970-03, 1983; George et al., J. Immunol. 141(6):2168-74, 1988; and Kwak, et al., Blood 76(11):2411-17, 1990).

The first attempts at bringing this idea and technology into the clinic were very labor intensive. These first attempts utilized mouse monoclonal antibodies generated against proteins isolated from the patients' individual lymphomas following biopsy. In one study, Meeker and coworkers generated mouse monoclonal anti-idiotype antibodies for treatment of a small group of patients after most had already undergone conventional lymphoma chemotherapeutic treatment (Meeker et al., Blood 65:1349-63, 1985). Positive results, however, were only obtained in roughly half the patients. Note also that lymphoma cells can develop resistance to such a treatment by a variety of means such as (a) switching the class of cell surface-expressed antibodies (Meeker et al., N Engl J Med. 312:1658-65, 1985), or (b) via a somatic mutation in the CDR2 region (Cleary et al., Cell 44:97-106, 1986). Thus, although this passive immunity approach for treatment has the advantage that it only requires isolation and purification of the relatively minor amount of a patient's idiotypic protein necessary for raising an antibody response in a mouse, its usefulness for treating lymphomas is nevertheless very limited.

Kwak et al. took a different approach and actively immunized patients using proteins purified from their own unique lymphomas. This approach suffers from the logistical requirement that large quantities of idiotypic proteins must be isolated (Kwak et al., N. Engl. J Med. 327:1209-15, 1992). Patients who had minimal or no disease following chemotherapy were treated by vaccination with autologous idiotype proteins. In order to obtain sufficient quantities of idiotypic proteins for vaccination, lymphoma cells obtained by biopsy were fused with an established tissue culture cell line to facilitate their growth in tissue culture, and the secreted idiotype proteins were purified via chromatography. However, large scale application of this method of treatment is precluded due to the extreme labor requirements, technical barriers, the requirement for a relatively large number of viable tumor cells, and prohibitive costs. Additionally, concerns have recently been raised concerning the viral loads associated with protein production in mammalian cells, which calls for increased caution in using such sources.

In a following paper, Hsu et al. reported on the phase I/II results of the above clinical trial utilizing vaccination of the idiotype conjugated to keyhole limpet hemocyanin (KLH) in the treatment of B-cell lymphoma (Hsu et al., Blood 89:3129-35, 1997). The results obtained showed that the generation of an anti-idiotype response correlated with improved clinical outcome. The duration of freedom from disease progression and overall survival of all patients mounting an anti-idiotype cellular immune response were significantly prolonged compared to those patients who did not mount an immune response. Again, to treat each individual patient, lymphoma cells obtained by biopsy were fused to established cell lines to allow the production of sufficient protein to immunize a typical patient. The results are promising, nevertheless this process is too difficult and impractical to use on a widespread or commercial scale.

More recently, Bendandi et al. demonstrated idiotypic, patient-specific immunization-induced remissions in patients with follicular lymphoma (Bendandi et al., Nat. Med. 5:1171-77, 1999). Following standard chemotherapy, twenty patients in complete clinical remission were immunized using patient-specific idiotypic proteins accompanied by granulocyte-monocyte colony-stimulating factor (GM-CSF; see infra.). Molecular analysis of the translocations characteristic of this lymphoma was conducted prior to chemotherapy, at clinical remission and following vaccination therapy. Eight of eleven patients with detectable translocations after chemotherapy-induced remission were found to undergo complete molecular remission following this vaccination. Tumor-specific cytotoxic CD8⁺and CD4⁺T cells were found in 19 of 20 patients. Tumor-specific antibodies were also detected but were not found to be required for remission. Again, this study used idiotypic proteins made up of the intact variable and constant region of the immunoglobulin found associated with the patient's lymphoma and produced by heterohybridoma fusion.

One avenue for developing more effective treatment regimens against B cell mediated malignancies is the integration of anti-CD20 therapy with other treatment methods. Such combination therapies could be other antibodies, other chemotherapeutic agents, or treatments directed at generally stimulating the patient's immune system. One such combination therapy is the use of anti-CD20 antibodies in combination with 20(S)-camptothecin such as is set forth in U.S. Pat. No. 6,420,378 B1 (issued on Jul. 16, 2002). Other such combination therapies are set forth in U.S. patent application No. 2001/0018041 A1 assigned to IDEC Pharmaceuticals Corp., published on Aug. 30, 2001 (using both anti-CD20 and anti-CD40L antibodies). These combination therapies may be useful as therapy for other types of lymphoma besides low grade, follicular non-Hodgkins lymphoma (NHL).

Unfortunately, however, cancer patients are frequently subject to a relapse of the disease following any treatment regimen. More effective or additional treatment would be beneficial, considering that in 1999 alone, close to 57,000 new cases of NHLs were predicted in the United States.

### SUMMARY OF THE INVENTION

The instant invention features the surprising synergistic effect of rapidly following treatment of B cell-mediated disease with a specific-binding cytoreductive agent, such as an anti-CD20 antibody, with an immunization with an idiotypic protein. Thus, in a first aspect, the invention features a method for treating a B cell malignancy in a patient with a combination therapy comprising contemporaneously co-administering (1) a specific-binding cytoreductive agent and (2) an immunotherapeutic composition comprising an autologous Id protein. For the purposes of this invention, contemporaneously co-administering refers to administering the immunotherapeutic composition at the beginning, during or at the end of the prior treatment or within three months of the end of the prior treatment.

In another aspect, the invention features a method for treating a B cell malignancy in a patient with a combination therapy comprising contemporaneously co-administering (1) a specific-binding cytoreductive agent and (2) an immunotherapeutic composition where the immunotherapeutic composition has been loaded into dendritic cells which thus contain at least part of the autologous Id protein. In a preferred embodiment of this aspect, the dendritic cells are autologous dendritic cells. Langerhans cells may be used in this aspect in a similar fashion to dendritic cells.

In a different aspect, the invention features a method for improving a treatment for a B cell malignancy wherein said treatment for a B cell malignancy comprises administrating a specific-binding cytoreductive agent and wherein said improved treatment comprises contemporaneously co-administering an autologous Id protein.

In preferred embodiments, the B cell malignancy has not been previously treated, or at least has not been previously treated with chemotherapeutic agents.

In other preferred embodiments, the disease to be treated is selected from the list consisting of relapsed B cell malignancy, B cell lymphoma, B cell leukemia, Hodgkin's Disease, a Non-Hodgkin's Lymphoma (wherein the Non-Hodgkin's Lymphoma is low grade, intermediate grade or high grade), small lymphocytic B-cell lymphoma, follicular and predominantly small cleaved cell B-cell lymphoma, follicular and mixed small cleaved and large cell type B-cell lymphoma, follicular and predominantly large cell type B-cell lymphoma, diffuse small cleaved cell B-cell lymphoma, diffuse mixed small and large cell B-cell lymphoma, diffuse large cell B-cell lymphoma, large cell immunoblastic B-cell lymphoma, lymphoblastic B-cell lymphoma, small non-cleaved Burkitt's and non-Burkitt's type B-cell lymphoma, AIDS-related lymphomas, angioimmunoblastic lymphadenopathy, monocytoid B-cell lymphoma, mantle cell lymphoma, chronic B-cell leukemia, acute lymphoblastic leukemia of a B-cell lineage, and chronic lymphocytic leukemia of a B-cell lineage.

In further embodiments, the contemporaneously co-administered treatment with autologous Id protein begins within three months of the last administration of said specific-binding cytoreductive agent, within two months of the last administration of said specific-binding cytoreductive agent, within one month of the last administration of said specific-binding cytoreductive agent, or within one week of the last administration of said specific-binding cytoreductive agent. The contemporaneously co-administered treatment with the autologous Id protein may also be simultaneously, or within one week, of the cytoreductive agent treatment.

In other embodiments, the specific-binding cytoreductive agent is an antigen-binding cytoreductive agent. In some of these embodiments, the antigen-binding cytoreductive agent is selected from the group consisting of an anti-CD20 antibody, an anti-CD52 antibody, an anti-CD22 antibody, an anti-B7 antibody, an anti-CD19 antibody, an anti-CD32 antibody, an anti-CD33 antibody, an anti-CD64 antibody, an anti-CD16 antibody, an anti-CD86 antibody, and an anti-CD156 antibody. Other specific-binding cytoreductive agents are also useful in the present invention. The cell surface of a malignant immune cell, such as a B cell, contains numerous proteins that might serve as ligand for a specific-binding cytoreductive agent. Such specific-binding cytoreductive agents capable of binding to malignant B cells and causing cytoreduction may also be used in this invention.

In further embodiments, the autologous Id protein is produced recombinantly in a host cell where potential host cells include mammalian, bacterial, yeast, insect, or other host cells. In other preferred embodiments, the autologous Id protein is produced recombinantly in insect cell lines. In further preferred embodiments the autologous Id protein is produced recombinantly in mammalial cell lines, including CHO cells and BHK cells. In still further preferred embodiments, the autologous Id protein is produced recombinantly in bacterial cells or yeast cells, including S. cerevisiae. In other preferred embodiments, the Id protein is delivered to the patient in the form of a DNA molecule encoding the Id protein, e.g., a DNA vaccine.

In some preferred embodiments, the autologous Id protein is coupled to KLH prior to administration.

In a related aspect, the autologous Id protein is encoded by an open reading frame wherein a portion of the DNA sequence of this open reading frame is obtained from a nucleic acid sequence isolated from the B cell malignancy of the patient to be treated. In one embodiment, the autologous Id protein does not comprise either a full length heavy antibody chain or a full length light antibody chain. In other embodiments, the Id protein does not comprise a fill length antibody heavy chain. In further embodiments, the autologous Id protein does not comprise a full length antibody light chain.

In another aspect of the present invention, the contemporaneously co-administered treatment with autologous Id protein begins before the end of administration of the specific-binding cytoreductive agent and wherein the treatment using the autologous Id protein and the cytoreductive agent continues in parallel. Thus, the treatment with the specific-binding cytoreductive agent continues after the first administration of the autologous Id protein, and the administration of the specific-binding cytoreductive agent and the administration of the autologous Id protein continue at intervals during the course of the treatment.

### BRIEF DESCRIPTION OF THE DRAWINGS

FIG. 1: FavId Treatment Following Cytoreduction with Rituxan in Follicular NHL Patients Relapsed/Refractory Following Chemotherapy. FIG. 1 shows the results of the trial as of this filing in Example 1 as compared to Witzig T. E., et al., “Randomized controlled trial of yttrium-90-labeled ibritumomab tiuxetan radioimmunotherapy versus rituximab immunotherapy for patients with relapsed or refractory low-grade, follicular, or transformed B-cell non-Hodgkin's lymphoma,” J Clin Oncol. (2002) May 15;20(10):2453-63. The graph compares the results of treatment with rituximab followed by FavId™ to the results of rituximab alone as published in Witzig, et al. These results demonstrate that treatment of chemotherapy relapsed/refractory B-cell NHL patients with FavId™ beginning two months following a single course of rituximab results in a significant prolongation of time to disease progression as compared to treatment with rituximab alone.

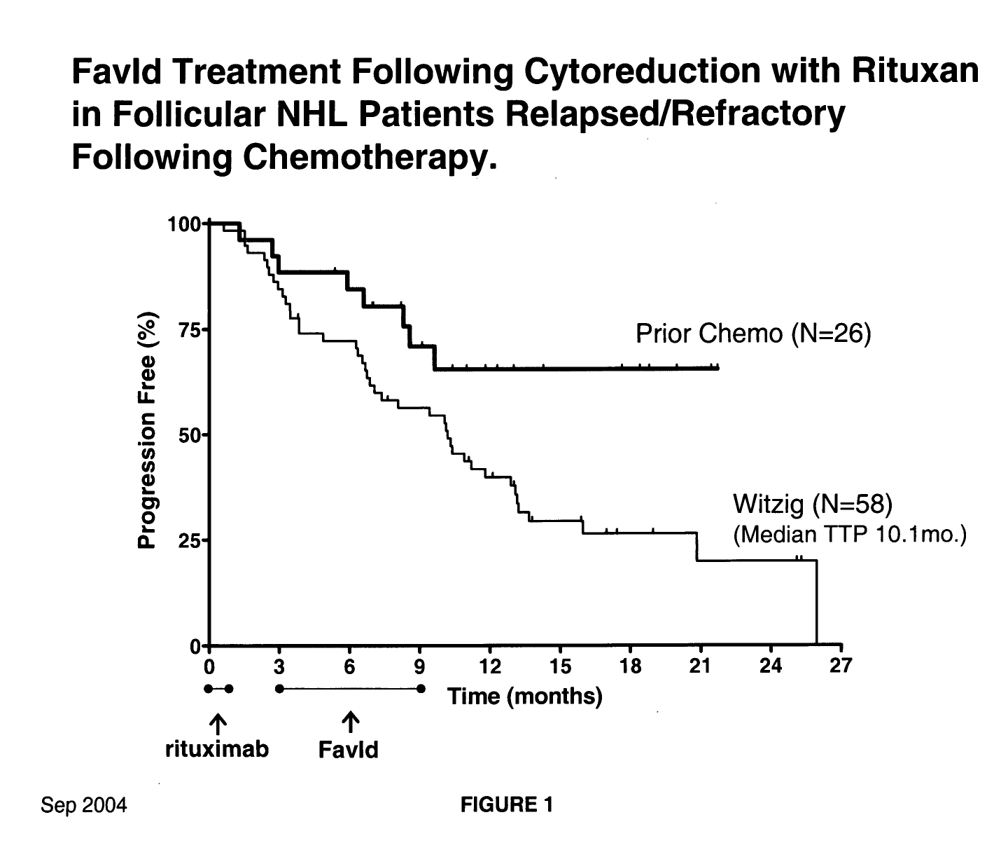

FIG. 2: FavId Treatment Following Cytoreduction with Rituxan in Treatment Naïve Follicular NHL Patients. FIG. 2 shows the results of the trial as of this filing in Example 2 as compared to Colombat P, et al. “Rituximab (anti-CD20 monoclonal antibody) as single first-line therapy for patients with follicular lymphoma with a low tumor burden: clinical and molecular evaluation,” Blood (2001) 97(1):101-106. These results demonstrate that treatment naïve B-cell NHL patients who received FavId™ two months following a single course of Rituximab have a significant prolongation of time to disease progression when compared to treatment with Rituximab.

FIG. 3: FavId Treatment Together with Cytoreduction with Rituxan in Treatment Naïve Follicular NHL Patients. FIG. 3 presents a comparison of the benefit of treatment with a single course of rituximab followed by treatment with FavId™ with the results presented in Hainsworth J D, et al. “Rituximab as First-Line and Maintenance Therapy for Patients With Indolent Non-Hodgkin's Lymphoma” J Clin Oncol. (2002) October 15;20:4261-4267. The data in Hainsworth et al. demonstrate the value of the regimen of rituximab administered every six months as indicated in FIG. 3. This is known to those practiced in the art as “maintenance Rituxan®” therapy, and is commonly accepted to result in a longer time to progression compared to a single course of Rituxan therapy for patients with indolent B-cell NHL. Maintenance Rituxan®” represents an improvement over the results of Colombat P, et al. Thus, a comparison of the results of a single treatment of rituximab plus FavId™ in treatment naïve B-cell NHL patients from Example 2 with the data of Hainsworth et al. suggests the potential for further benefit to the patients if FavId™ is combined with maintenance therapy of Hainsworth et al. if the overall suppression of immune system does not counteract the benefit of FavId™. The protocol for this trial is set forth in Example 8.

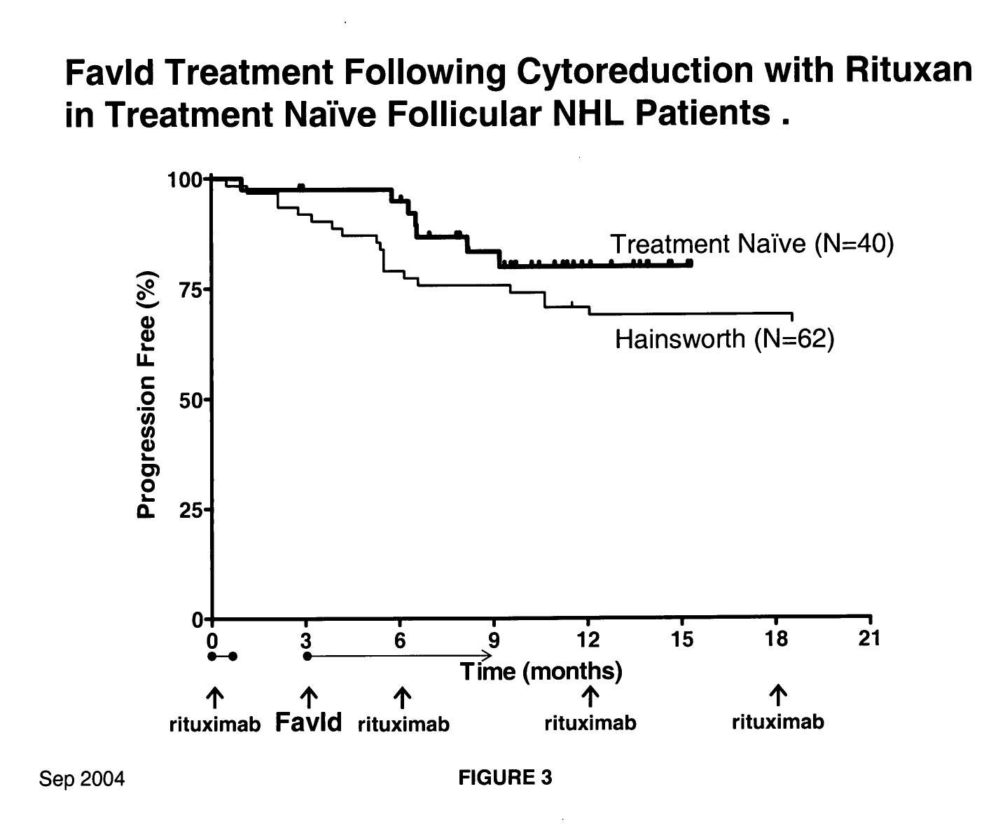

### DETAILED DESCRIPTION OF THE INVENTION

The present invention features the surprising combination of a specific binding cytoreductive agent, such as an anti-CD20 antibody, soon followed by immunization with an idiotypic protein. In previously used therapy, one regimen of treatment, such as chemotherapy, is followed by a period of recovery and remission before a relapse forces the next round of therapy. Additionally, if immunization using an idiotypic protein was contemplated, the treating physician might be inclined to wait for the immune system to recover from the previous treatment before trying to induce an immune response. This would be especially true if treatment with an anti-CD20 antibody has specifically depleted the overall B cell population in this patient. Immediately or contemporaneously co-administering an immunogen in the form of an idiotypic protein would not induce a substantial antibody response directed at the idiotypic protein. The present inventors, however, discovered a surprising synergistic effect by rapidly following treatment with a specific binding cytoreductive agent, such as an anti-CD20 antibody, e.g. rituximab, with immunization with an idiotypic protein, despite the anticipated deletion of the B cell/antibody response arm of the immune response. Without being bound by this or any other theory, one advantage of the present invention is that cytoreduction with a specific-binding agent followed rapidly with an active immunotherapeutic agent in which the intended target of the immunotherapeutic agent is found within the cytoreduced population results in a potent antitumor immune response that primarily depends on T cells for its therapeutic effect.

The invention also features the administration of a specific-binding cytoreductive agent and an autologous Id protein in an overlapping time course, where the administration of the autologous Id protein first occurs before the end of the treatment with the specific-binding cytoreductive agent, and the treatment with both agents continue in a coordinated manner. One embodiment of an overlapping time course is when a specific-binding cytoreductive agent is administered every six months while the autologous Id protein is administered every month, but not during the same month as the specific-binding cytoreductive agent is administered. The specific-binding cytoreductive agent may also be administered about every two weeks, about every three weeks, about every month, about every two months, about every three months, about every four months, or about every six months. The autologous Id protein may be administered about every two weeks, about every three weeks, about every month, about every two months, about every three months, about every four months, or about every six months. In some embodiments, the schedule of administration for either the specific-binding cytoreductive agent or the autologous Id protein may be interrupted for the administration of the other.

Combining treatments using a specific binding cytoreductive agent has further advantages. It is well accepted to those of skill in the art that immune therapy of cancers is most likely to be effective in the residual disease setting following a reduction in the bulk of the cancer. An advantage of following specific binding cytoreductive agent therapy with targeting the Id protein is that the Id protein uniquely expressed by a patient's B cell malignancy is not found in elsewhere in the body. Therefore, generating an immune response against a lymphoma's Id protein will not result in significant toxicity to normal tissue. Furthermore, such an active immune response provoked by using the Id protein as an immunogen should be long lived and therefore delay or prevent the recurrence of the disease. Finally, active immune responses provide a broader immune response than that seen with passive antibody therapy such as using rituximab or similar agents. This active immune response driven by immunization with the Id protein may be able to better neutralize tumor heterogeneity and mutational drift in a B cell malignancy's Id protein sequence over time.

Definitions

The term “treating” refers to administering a composition to an organism afflicted with an abnormal condition, such as a B cell malignancy, where the administration of the composition has a therapeutic effect and at least partially alleviates or abrogates the abnormal condition. Note that the treatment need not provide a complete cure and the treatment will be considered effective if at least one symptom is improved or eradicated. Furthermore, the treatment need not provide a permanent improvement of the medical condition or other abnormal condition, such as a B cell malignancy, although this is preferable. The treatment may work by killing the cancerous cells, facilitating the killing of the cancerous cells, inhibiting the growth of the cancerous cells, and/or inhibiting the process of the metastasis of the cancer.

The term “patient” refers to an organism that has presented at a clinical setting with a particular symptom or symptoms suggesting the need for treatment with a therapeutic agent. The treatment may either be generally accepted in the medical community or it may be experimental. In preferred embodiments, the patient is a mammal, including animals such as dogs, cats, pigs, cows, sheep, goats, horses, rats, and mice. In further preferred embodiments, the patient is a human. Note that a patient's diagnosis can alter during the course of disease progression, either spontaneously or during the course of a therapeutic regimen or treatment, nevertheless, such a patient can still be considered in need of treatment.

The term “therapeutic effect” refers to the inhibition or activation of factors causing or contributing to an abnormal condition (including a disease or disorder). A therapeutic effect relieves or prevents to some extent one or more of the symptoms of the abnormal condition. In reference to the treatment of abnormal conditions associated with the present invention, a therapeutic effect can refer to one or more of the following: (a) an increase or decrease in the number of cells present at a specified location or within an organism, such as an overall decrease of lymphoma cells; (b) an increase or decrease in the ability of cells to migrate; (c) an increase or decrease in the proliferation, growth, and/or differentiation of cells; (d) an inhibition (i.e., slowing or stopping) or acceleration of cell death, such as increasing the rate of apoptosis of lymphoma cells; (e) relieving, to some extent, one or more of the symptoms associated with the abnormal condition; (f) enhancing or inhibiting the function of an affected population of cells, such as activating T cells.

The term “therapeutically effective amount” as used herein means that amount of active compound that elicits a biological or medicinal response in a tissue, system, animal or human that is being sought by a researcher, veterinarian, medical doctor or other clinician in order to provide a therapeutic effect.

The term “abnormal condition” refers to a function in the cells or tissues of an organism that deviates from their normal functions in that organism and includes, but is not limited to, B-cell malignancies. An abnormal condition can relate to cell proliferation, cell differentiation, cell survival, cellular function, or the activities of enzymes within a cell. Abnormal conditions relating to cell proliferative disorders include cancers such as B-cell malignancies, B-cell lymphomas, B-cell leukemias, T-cell malignancies, T-cell lymphomas, and T-cell leukemias. Abnormal conditions relating to cell survival refer to conditions in which programmed cell death (apoptosis) pathways are activated or abrogated.

The terms “administration” or “administering” refer to a method of incorporating a compound into the cells or tissues of an animal, preferably a mammal, in order to treat or prevent an abnormal condition or B-cell malignancy. When the compositions of the invention are provided as combinations of active agents, the terms “administration” and “administering” include sequential or concurrent introduction of the compositions with the other agent(s). For cells harbored within the organism, many techniques exist in the art to administer compounds. The therapeutic composition may be administered by any conventional route, including injection or by gradual infusion over time. The administration may, depending on the composition being administered, for example, be oral, pulmonary, intravenous, intraperitoneal, intramuscular, intracavity, subcutaneous, or transdermal. A compound may also be administered to an organism by isolating or withdrawing cells from the organism, administering a compound to the isolated or withdrawn cells, and returning the cells to the organism. For cells outside of the organism, and in a few cases within the organism, multiple techniques exist in the art to administer the compositions, including (but not limited to) cell microinjection techniques, transformation techniques, incubation, and carrier techniques.

The term “contemporaneously co-administering” refers to administering two or more treatment regimens at linked times where the second treatment regimen may begin at the end of the first treatment regimen or within up to three months after the end of the first treatment regimen. Thus, if a hypothetical Regimen A ended on March 1st, the contemporaneously co-administered Regimen B could begin somewhere during the period from March 1 st to May 31 st of the same year. In other embodiments of the instant invention, “contemporaneously co-administering” refers to beginning the treatment regimen with the autologous Id protein simultaneously, during, or within three months of the end of the treatment of the specific-binding cytoreductive agent. In the methods of the invention, an autologous Id protein may be administered simultaneously with the cytoreductive agent, within one month, two months, three months, or at any weekly subinterval such as one, two, three, four, five, six, seven, eight, nine, ten, eleven or twelve weeks since the last administration of the last specific-binding cytoreductive agent.

In further embodiments, the autologuous Id protein and the cytoreductive agent may be administered in an overlapping fashion where the administration of the autologous Id protein begins before the last administration of the cytoreductive agent, and administration of the two continues in parallel. Thus, the administration of an autologous Id protein may be followed by administration of a specific-binding cytoreductive agent which is in turn followed by administration of an autologous Id protein; and this pattern of overlapping administration may continue. In some embodiments, the specific-binding cytoreductive agent may be administered about every two weeks, about every three weeks, about every month, about every two months, about every three months, about every four months, or about every six months. The autologous Id protein may be administered about every two weeks, about every three weeks, about every month, about every two months, about every three months, about every four months, or about every six months. The schedule of administration of either the specific-binding cytoreductive agent or the autologous Id protein may be interrupted for the administration of the other. The administration of the specific-binding cytoreductive agent or the autologous Id protein need not follow a regular schedule and may be administered at variable intervals.

In preferred embodiments, the first regimen utilizes a specific-binding cytoreductive agent. In other preferred embodiments, the first regimen utilizes an anti-CD20 antibody. In further preferred embodiments, the first regimen utilizes rituximab or tositumomab. In preferred embodiments, the second regimen utilizes treatment with an idiotypic protein.

One example of a specific-binding cytoreductive agent treatment regimen is a course of treatment with an anti-CD20 monoclonal antibody. In a similar fashion to “contemporaneously co-administering,” the terms “co-treating,” “proximately linked treatment,” “proximately administering treatment,” or “temporally-linked treatment” as used herein may also refer to two treatment regimens being conducted simultaneously, or in an overlapping fashion, or so that the second regimen begins within three months following the last administration of the last apoptotic agent. Note also that these terms also refer to treatment where, if the two treatment regimens overlap, then the first treatment regimen is not finished before the second begins.

In certain embodiments, the term “contemporaneously co-administering” may refer to an autologous Id protein being administered simultaneously, within one month, two months, three months, or any weekly subinterval such as one, two, three, four, five, six, seven, eight, nine, ten, eleven or twelve of the last administration of the last specific-binding cytoreductive agent. In preferred embodiments, the first regimen utilizes a specific-binding cytoreductive agent. In other preferred embodiments, first regimen utilizes an anti-CD20 antibody. In further preferred embodiments, first regimen comprises rituximab or tositumomab. In still further preferred embodiments, the specific-binding cytoreductive agent is selected from the group consisting of an anti-CD52 antibody, an anti-CD22 antibody, an anti-B7 antibody, an anti-CD19 antibody, an anti-CD32 antibody, an anti-CD16 antibody, an anti-CD86 antibody, and an anti-CD156 antibody. Other specific-binding cytoreductive agents are also useful in the present invention. The cell surface of a malignant immune cell, such as a B cell, contains numerous proteins that might serve as ligand for a specific-binding cytoreductive agent. Such specific-binding cytoreductive agents capable of binding to malignant B cells and causing cytoreduction may also be used in this invention. In preferred embodiments, the second regimen utilizes treatment with an idiotypic protein.

In another embodiment, the autologuous Id protein and the cytoreductive agent may administered in an overlapping fashion where the administration of the autologous Id protein begins before the last administration of the cytoreductive agent, but after the first administration of the cytoreductive agent, and administration of the autologuous Id protein and the cytoreductive agent continues in a parallel.

The term “specific-binding cytoreductive agent” refers to a cytoreductive agent that selectively and specifically binds to its designated target, possibly a cell-surface antigen, and thereby causes cytoreduction by killing or inhibiting the growth of the targeted cell or killing the target cell population. In preferred embodiments, a “specific-binding cytoreductive agent” is an antigen-binding cytoreductive agent. In further preferred embodiments, a specific-binding cytoreductive agent is an anti-CD20 monoclonal antibody such as rituximab. In other preferred embodiments, a specific-binding cytoreductive agent also may be an antibody that specifically binds to different surface antigens, such as CD19, CD22, B7, or CD156. A specific-binding cytoreductive agent may be an antibody, or it may be a functional derivative of an antibody such as a ScFv, a camelid antibody fragment, or other functional derivatives of antibodies. A cytoreductive agent may be an apoptotic agent. Other specific-binding cytoreductive agents are possible. Bi-specific antibodies, capable of binding more than one cell-surface antigen are also useful in the present invention. Further, the cell surface of a malignant immune cell, such as a B cell, contains numerous proteins that might serve as ligand for a specific-binding cytoreductive agent. Such specific-binding cytoreductive agents capable of binding to malignant B cells and causing cytoreduction may also be used in this invention.

The term “cytoreductive agent” refers to an agent that either reduces cell number or slows cell division in the target population of cells. A cytoreductive agent may act through inducing apoptosis, but other mechanisms are possible.

When the compounds of the present invention are administered to a subject, they are generally prepared as “compositions.” Such compositions are frequently mixtures and may routinely contain pharmaceutically acceptable concentrations of salts, buffering agents, preservatives, compatible carriers, and optionally other therapeutic agents. A composition may include carrier proteins such as a KLH and adjuvants such as a KLH, BCG, aluminum salts, and MF59 (Singh and O'Hagen, Nat. Biotech 17:1075-81 (1999)), as well as stimulatory nucleic acid compounds such as CpG oligonucleotides (see e.g., Jahrsdorfer B, et al., J Leukoc Biol. 69(1):81-8 (2001); Krieg A M, et al., Trends Microbiol. 6(1):23-7 (1998)). Components of the composition may be covalently bound to the autologous Id protein. A composition may include cytokines such as GM-CSF and MCP-3. The therapeutic composition may be administered by any conventional route, including injection or by gradual infusion over time. The administration may, depending on the composition being administered, for example, be oral, pulmonary, intravenous, intraperitoneal, intramuscular, intracavity, subcutaneous, or transdermal.

The term “autologous” as used herein refers to the amino acid sequence for the immunoglobulin's variable region or the nucleic acid sequence for a gene encoding the patient's immunoglobulin's variable region being derived from the patient to be treated. The protein may be subsequently synthesized in vitro, and the nucleic acid sequence may be synthesized, manipulated, or assembled in vitro, nevertheless the sequences of the variable region of the Id protein are associated in the patient to be treated with the patient's malignancy the variable region sequences are derived from biopsies taken from the patient. The exact combination of variable regions and immunoglobulin subtype used in the idiotype protein may not be expressed by the patient's malignancy, but the constant region of the autologous Id protein is found generally in humans and the variable region is derived from the patient. In reference to dendritic cells, or other cells derived from an organism, the term “autologous” has a further meaning. “Autologous” cells are obtained and/or isolated from the patient to be treated. Thus, autologous dendritic cells are obtained from the patient to be treated; in preferred embodiments, the autologous dendritic cells are contacted with the autologous Id protein, and then administered back to the patient.

The term “Id protein” or “autologous Id protein” as used herein refers to the autologous tumor-derived idiotypic surface Ig derived from a given patient, also known as the idiotype protein. In preferred embodiments, the Id protein is derived from a patient's B cell tumor, a patient's B cell malignancy, or cells derived from the tumor or malignancy.

The term “recombinant autologous Id protein” refers to an autologous Id protein which has been synthesized outside of the patient by recombinant means. In preferred embodiments, the recombinant autologous Id protein has been synthesized in insect cells. In other preferred embodiments, the recombinant autologous Id protein has been synthesized in yeast or bacterial cells. In further preferred embodiments, the recombinant autologous Id protein has been synthesized in mammalian cells. The Id protein administered to a patient is generally a chimeric protein as the final sequence is derived both from the patient and the expression vector. In some cases, the amino acid sequence of the recombinant autologous Id protein may be identical to the Id protein found in the patient.

The term “contacting” as used herein refers to adding together a solution or composition comprising the Id protein with a liquid medium bathing a polypeptide or cell. The solution comprising the Id protein may also comprise another component used to facilitate the uptake of the Id protein into the cells of the methods. The solution comprising the test compound may be added to the medium bathing the cells by utilizing a delivery apparatus, such as a pipette-based device or syringe-based device.

The term “primary malignancy” as used herein refers to B cell malignancy that has not been previously treated by chemotherapy before commencing therapy with a specific-binding cytoreductive agent. Similarly, the term “untreated” refers to a B cell malignancy from a patient that has not been treated for the B cell malignancy prior to undergoing therapy by the means of the instant invention. For example, such a patient has not undergone a course of chemotherapy.

The term “apoptotic agent” as used herein is a compound, drug, toxin, composition, or, rarely, a biological entity which begins or promotes the process of apoptosis, or programmed cell death, in a cell. In preferred embodiments, the cell in which apoptosis is began or promoted is a cell of a B cell malignancy. Exemplary apoptotic agents include, but are not limited to, TNF-α, AIM I (see, International Publication No. WO 97/33899), AIM II (see, International Publication No. WO 97/34911), and Fas Ligand (Takahashi et al., Int. Immunol., 6:1567-1574 (1994)), VEGI (see, International Publication No. WO 99/23105).

The term “recombinantly” as used herein can refer to a means of producing a compound by first transcribing, then translating a DNA sequence that has been linked to other DNA sequences than which it is associated in nature. A recombinant product can be a peptide, a polypeptide, a protein, an enzyme, an antibody, an antibody fragment, a polypeptide that binds to a regulatory element, a structural protein, an RNA molecule, and/or a ribozyme, for example. In preferred embodiments, the recombinant product of the instant invention is an idiotype protein. In other preferred embodiments, the recombinant product of the instant invention is a monoclonal antibody. In further preferred embodiments, the recombinant product of the instant invention is an antibody analog.

This list of recombinant products is for illustrative purposes only and the invention may relate to other types of recombinant products. The DNA sequence used as a source for the production of the gene product is generally obtained in whole or in part from another organism or cell than the one used for the expression, then ligated into an expression vector for producing the gene product in a recombinant fashion. Note that human genes may be recombinantly expressed in a human cell line if the human gene is first isolated, then inserted into an expression vector, then returned to a human cell.

The terms “protein,” “polypeptide,” and “peptide” are used herein interchangeably and are used in their conventional meaning, i.e., a chain of amino acids. None of the terms imply any set length or range of lengths of amino acid chains. These terms also do not imply or exclude post-translational (or post-synthetic) modifications of the polypeptide, e.g., glycosylations, acetylations, phosphorylations, and the like, as well as other modifications known to those of skill in the art, both naturally occurring and non-naturally occurring. A polypeptide may be an entire protein, or a region of the amino acid chain. A protein may comprise more than one polypeptide.

The term “insect cell lines” refers to cell lines derived from insects. Many such insect cell lines are susceptible to infection by the baculovirus. One skilled in the art is familiar with such cell lines and the techniques needed to utilize them. Representative examples of insect cell lines include Spodoptera frugiperda (sf9) and Trichoplusia ni cell lines (see U.S. Pat. Nos. 5,300,435 and 5,298,418).

The terms “Trichoplusia ni cells” and “Spodoptera frugiperda cells” refers to insect cell lines frequently used in combination with baculovirus expression vectors. One skilled in the art is familiar with these cell lines and how to obtain them.

The term “vector” herein may refer to a DNA molecule into which another DNA molecule of interest, such as a molecule encoding an Id protein, can be inserted into the DNA of the vector. Examples of classes of vectors can be plasmids, cosmids, viruses, and bacteriophages (‘phages’). Typically, vectors are designed to accept a wide variety of inserted DNA molecules and then used to transfer or transmit the DNA of interest into a host cell (e.g., bacterium, yeast, higher eukaryotic cell). A vector may be chosen based on the size of the DNA molecule to be inserted, as well as based on the intended use. For transcription into RNA or transcription followed by translation to produce an encoded polypeptide, an expression vector may be chosen. For the preservation or identification of a specific DNA sequence (e.g., one DNA sequence in a cDNA library) or for producing a large number of copies of the specific DNA sequence, a cloning vector may be chosen, although expression vectors may also be used for cloning or storage. If the vector is a virus or bacteriophage, the term vector may include the viral or bacteriophage coat.

Following entry into a cell, all or part of the vector DNA, including the insert DNA, may be incorporated into the host cell chromosome, or the vector may be maintained extrachromosomally. Those vectors that are maintained extrachromosomally are frequently capable of autonomous replication in a host cell into which they are introduced (e.g., many plasmids having a bacterial origin of replication). Other vectors are integrated into the genome of a host cell upon introduction into the host cell, and thereby are replicated along with the host genome.

The term “expression vector” as used herein may refer to a DNA construct which allows one of skill in the art to place a gene encoding a gene product of interest, usually a protein, into a specific location in a vector from which the selected gene product can be expressed by the machinery of the selected host cell, or alternately, by an in vitro expression system. This type of vector is frequently a plasmid, but other forms of expression vectors, such as bacteriophage vectors and viral vectors (e.g., adenoviruses, replication defective retroviruses, and adeno-associated viruses), may be employed. The selection of expression vectors, control sequences, transformation methods, and the like, are dependent on the type of host cell used to express the gene. An expression vector of the invention may use the p10 promoter, the polyhedrin promoter, the honey bee melittin signal sequence or the human alkaline phosphatase signal sequence. Such an expression vector may use these, or other, promoters or signal sequences in any combination. Thus, the p10 promoter may be operatively linked to either the honey bee melittin signal sequence or the human alkaline phosphatase signal sequence, and the polyhedrin promoter may be linked to either the honey bee melittin signal sequence or the human alkaline phosphatase signal sequence. The same promoter may be operatively linked to both genes encoding the chimeric proteins in an expression vector. The same signal sequence may be operatively linked individually to both genes encoding the chimeric proteins in an expression vector.

The term “baculovirus expression vector” refers to a DNA construct which is designed to express an inserted gene of interest when used in the baculovirus system where expression usually is conducted in insect cells. Any of the potential baculoviruses or expression vectors designed to function in the baculovirus system may be used in the instant invention. In a similar fashion, the term “expression vector” is a genus which encompasses the particular embodiment of baculovirus expression vectors, but “expression vectors” may function in cells and cell lines aside from, or in addition to, insect cell lines.

The term “open reading frame” or “ORF” refers to a region of a polynucleotide sequence which encodes a polypeptide. This region may represent a portion of a coding sequence or a total coding sequence. In preferred embodiments, in refers to the entire sequence between the start codon and the stop codon.

The term “derived from a patient” refers to genetic materials or cells which have been isolated or purified from a sample of blood or tissue obtained from a patient. The term “derived from a B cell malignancy” refers to genetic material which has been isolated or purified from a sample of a B cell malignancy obtained from a patient.

The term “full length heavy chain” refers to the sequence of the intact cDNA molecule or the intact heavy chain immunoglobulin polypeptide chain as isolated from the B cell malignancy of a patient. The full length heavy chain may be any subtype immunoglobulin chain.

The term “full length light chain” refers to the sequence of the intact cDNA molecule or the intact light chain immunoglobulin polypeptide chain as isolated from the B cell malignancy of a patient. Such a full length light chain may be either a κ or λ chain.

The term “chimeric protein” refers to a protein which comprises a single polypeptide chain comprising segments derived from at least two different proteins. The segments of the chimeric protein must be derived from heterologous proteins, that is, all segments of the chimeric polypeptide do not arise from the same protein as occurs in nature. The chimeric proteins such as used in the present invention include proteins containing portions of the V_{H} or V_{L} region of an immunoglobulin chain, but where the chimeric proteins do not comprise the entire C region of those chains as found in the B cell clone from which the V_{H} or V_{L} region is derived. Furthermore, the V_{H} or V_{L} region may not necessarily include the entire variable region, but does include enough to generate an immune response. Chimeric proteins as used in the present invention may also include proteins in which a segment of the naturally occurring protein has been replaced with an equivalent naturally or non-naturally occurring segment. This includes replacing the IgG₁ constant region derived from a patient with the IgG₁ constant region from a different source, and also includes immunoglobulin constant regions in which a segment of the protein has been replaced with a linker, segment or domain that is partially or entirely manmade. In some cases, however, the gene or the coding region for the chimeric protein of the instant invention may be the same as the gene for the immunoglobulins that occur naturally in the patient. The gene or open reading frame for the chimeric protein may be distinguishable from naturally occurring protein's open reading frame for one or more of the following reasons: (1) it will be the full length immunoglobulin gene or cDNA from the patient, or some smaller part of the immunoglobulin gene or cDNA; (2) it will be a different subtype than isolated from the patient; or (3) the nucleic acid sequence encoding the patient's IgG₁ constant region will differ from the portion of the IgG₁ gene used in the expression vector; and/or (4), the gene used for idiotype expression will not contain introns identical to those expressed in the messenger pre-mRNA in the B cell malignancy. Chimeric proteins may also be referred to as “fusion proteins.”

The term “segment” or “portion” or “part” is used to indicate that section of a polypeptide derived from the amino acid sequence of a polypeptide having a length less than the full-length polypeptide from which it has been derived. It is understood that such segments may retain one or more characterizing portions of the native polypeptide. Examples of such retained characteristics include: binding with an antibody specific for the native polypeptide, or an epitope thereof.

The terms “V_{H}” and “V_{L}” refer to the variable regions of the polypeptide chains of immunoglobulin molecules, or nucleic acids encoding such polypeptide chains as is understood by one skilled in the art. The exact sequence of a variable region cannot be predicted and must be determined by isolating the sequence (protein or gene) in question. The V_{H} and V_{L} regions isolated from particular patients are used in the instant invention. The exact sequence of a kappa (κ) or lambda (λ) light chain is determined by clonal rearrangements of the V regions, J regions and Constant region of the light chain locus. (The kappa and lambda loci are separate and distinct.) The exact sequence of a heavy chain is determined by clonal rearrangements of the V regions, D regions, J regions and Constant region of the heavy chain locus. Additional sequence variation in the variable region arises from imprecision during the recombination process and also is generated by somatic mutations subsequent to the end of the recombination process. The terms “V_{H}” and “V_{L}” also refer to portions or segments of the V_{H} and V_{L} regions. A segment of the V_{H} or V_{L} region may also include all or substantially all of the V region. The term “substantially all” refers to approximately 95%, 90%, 85%, 80%, 75%, 70%, 65%, 60%, 55%, 50%, or less, of the entire variable region. The portion of the V_{H} or V_{L} region present must be sufficient to allow the chimeric molecule to operate in the present invention. The terms “V_{H}” and “V_{L}” may also refer to functional derivatives of such polypeptide regions.

The term “immunoglobulin constant region” refers to all or part of that portion of immunoglobulin molecules which are not encoded by the variable regions of immunoglobulins. The term “immunoglobulin constant region” may also refer to the DNA sequence encoding the immunoglobulin constant region. The immunoglobulin constant region includes the segments CL, CH1, CH2, CH3, and the Hinge region. Immunoglobulin types include IgG_{γ1}, IgG_{γ2}, IgG_{γ3}, IgG_{γ4}, IgA₁, IgA₂, IgM, IgD, IgE heavy chains, and κ or λ light chains or segments thereof. Any immunoglobulin constant region segments may be used for chimeric Id proteins. Functional derivatives of the immunoglobulin constant region segments may also be used.

The terms “IgG₁, IgG₂, IgG₃, IgG₄, IgA, IgA₁, IgA₂, IgM, IgD, IgE” refer to classes and subclasses of human immunoglobulins. The terms may refer to either the DNA sequences, RNA sequences or the amino acid sequences of the proteins. The class and subclass of an immunoglobulin molecule is determined by its heavy chain. IgG and IgD are different classes of immunoglobulins; IgG₁ and IgG₂ are different subclasses of immunoglobulin molecules. The term “IgA” may refer to any subclass of IgA molecules. In preferred embodiments, it refers to an IgA₁ molecule. In other preferred embodiments, it refers to an IgA₂ molecule. In some embodiments, the immunoglobulin heavy chain used may be a chimeric protein that contains amino acids from a second protein. The term “IgG_{γ1}” refers to the heavy chain associated with the IgG₁ class of immunoglobulins. IgG₁ represents approximately 66% of human IgG immunoglobulins (Roitt et al., Immunology, Mosby, St. Louis, pg. 4.2, 1993).

The terms “kappa constant region,” “lambda constant region,” “κ constant region,” and “λ constant region” refer to the constant regions of kappa (κ) and lambda (λ) light chains that remain constant during the development of the immune system. The terms may refer to either the DNA sequences, RNA sequences or the amino acid sequences of the proteins. In some embodiments of the present invention, portions of the immunoglobulin light chain may be comprised in a chimeric protein that contains amino acids from one or more other proteins.

The term “isolating” refers to removing a naturally occurring nucleic acid sequence from its normal cellular environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes. Also, by the use of the term “isolating” in reference to nucleic acid is meant that the specific DNA or RNA sequence is increased to a significantly higher fraction (2- to 5-fold) of the total DNA or RNA present in the solution of interest than in the cells from which the sequence was taken. This enrichment could be caused by a person by preferential reduction in the amount of other DNA or RNA present, or by a preferential increase in the amount of the specific DNA or RNA sequence, or by a combination of the two. However, it should be noted this “enrichment” does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid. The term “isolating” may also include a step wherein an RNA sequence is converted into a cDNA sequence by techniques well known in the art.

Isolated DNA sequences are relatively more pure than in the natural environment (compared to the natural level this level should be at least 2- to 5-fold greater, e.g., in terms of mg/mL). Individual sequences obtained from PCR may be purified to electrophoretic homogeneity. The DNA molecules obtained from this PCR reaction could be obtained from total DNA or from total RNA. These DNA sequences may or may not be naturally occurring, but rather are preferably obtained via manipulation of a partially purified naturally occurring substance (e.g., messenger RNA (mRNA)). For example, the construction of a cDNA library from mRNA involves the creation of a synthetic substance (cDNA) and pure individual cDNA clones can be isolated from the synthetic library by clonal selection from the cells carrying the cDNA library. The process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

The term “gene encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a DNA molecule, it may comprise sequences necessary or helpful for directing the expression of the gene. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF).

The term “inserting” refers to a manipulation of a DNA sequence via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The term “allow the expression of” refers to placing an expression vector into an environment in which the gene of interest will be expressed. This commonly means inserting the expression vector into an appropriate cell type where the promoter and other regions necessary for gene expression will be recognized by the host cell's components and will cause the expression of the gene of interest. The expression normally consists of two steps: transcription and translation. Expression can also be conducted in vitro using components derived from cells. One skilled in the art is familiar with these techniques, and such techniques are set forth in Sambrook et al. (Sambrook, Fritsch, & Maniatis, “Molecular Cloning: A Laboratory Manual”, 2nd ed., Cold Spring Harbor Laboratory, 1989). In the preferred embodiment, the expressed product is a protein or polypeptide. In other preferred embodiments, the expressed product is V_{H}/IgG_{γ1}, V_{L}/C_{κ}, V_{L}/C_{λ}, or V_{L}/IgG_{γ1}.

The term “secretory signal sequence” refers to a peptide sequence or a nucleotide sequence directing the export of a protein from the cell. When this nucleotide sequence is translated in frame as a peptide attached to the amino-terminal end of a polypeptide of choice, the peptide secretory signal sequence will cause the secretion of the polypeptide of choice by interacting with the machinery of the host cell. As part of the secretory process, this secretory signal sequence will be cleaved off, leaving only the polypeptide of interest after it has been exported. In preferred embodiments, the honey bee melittin secretory signal sequence is employed. In other preferred embodiments, the human placental alkaline phosphatase secretory signal sequence is employed. Baculovirus expression systems are not limited by these secretory signal sequences and others known to those skilled in the art may be substituted in place of, and in addition to, these.

The term “promoter controls” refers to an arrangement of DNA in an expression vector in which a promoter is placed 5′ to a gene of interest and directs the transcription of the DNA sequence into an mRNA molecule. This mRNA molecule can be then translated by the host cell's machinery.

The terms “protein A,” “protein G,” and “protein L” refer to specific bacterial proteins which are capable of specifically binding immunoglobulin molecules without interacting with an antigen binding site. Protein A is a polypeptide isolated from Staphylococcus aureus that binds the Fc region of immunoglobulin molecules. Protein G is a bacterial cell wall protein with affinity for immunoglobulin G (IgG), which has been isolated from a human group G streptococcal strain (G148). Protein L is an immunoglobulin light chain-binding protein expressed by some strains of the anaerobic bacterial species Peptostreptococcus magnus.

The term “operatively linked” refers to an arrangement of DNA in which a controlling region, such as a promoter or enhancer, is attached to a connected DNA gene of interest so as to bring about its transcription, and hence allowing its translation. The term “operatively linked” may also refer to a DNA sequence encoding a processing signal, such as a secretory signal sequence, connected to a gene encoding a polypeptide to form a single open reading frame. Following transcription and translation, the secretory signal sequence has the potential to bring about the export of the translated polypeptide.

The term “isolating” as refers to a protein or polypeptide, refers to removing a naturally occurring polypeptide or protein from its normal cellular environment or refers to removing a polypeptide or protein synthesized in an expression system (such as the baculovirus system described herein) from the other components of the expression system. Thus, the polypeptide sequence may be in a cell-free solution or placed in a different cellular environment. The term does not imply that the polypeptide sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present (or other material), or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term significant here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. In preferred embodiments, the amino acid sequence is a chimeric protein as described above. The term is meant to cover only those situations in which a person has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

The term “chemotherapeutic agent” as used herein is a compound, drug, toxin, or composition which is used to induce a therapeutic effect in a patient suffering from cancer. In preferred embodiments, the cancer is a B cell malignancy. A variety of possibilities for chemotherapeutic agents are listed herein and these, as well as others, are well known in the art. Also available are biotech therapeutic agents such as rituximab and tositumomab.

The term “chemotherapeutic agent” as used herein also includes, for example, DNA-interactive agents, antimetabolites, tubulin-interactive agents, hormonal agents and others, such as asparaginase or hydroxyurea. Chemotherapeutic agents maybe grouped as DNA-interactive agents, antimetabolites, tubulin-interactive agents, hormonal agents, and other agents such as asparaginase or hydroxyurea. Each type of chemotherapeutic agents can be further subdivided. Chemotherapeutic agents may be selected from any of these groups but are not limited thereto.

Immunotherapy Using a Patient's Own Idiotype Produced in Insect Cells

Historically, surface antigens of circulatory cells and those of the immune system have been extensively characterized. This vantage point has led to the realization that many cancers of the circulatory system have clonally unique surface antigens; B cell tumors, especially, consist of mass-produced cells expressing a single antibody with that antibody presented on the cell's surface as the idiotype protein. This immediately suggests that a B cell tumor could be selectively attacked using the precision of the immune system rather than resorting to the mass destruction of chemotherapy.

The possibility of evoking an immune response that would recognize and eliminate only neoplastic cells while sparing normal tissue represents an exciting approach to the treatment of cancer. As seen above in the Background of the Invention, impractical means of accomplishing this have been developed. Inducing such an immune response requires identifying a unique tumor antigen and presenting it as an immunogen. Most B cell malignancies arise from the clonal expansion of a single B cell, thus each B-cell harbors a unique genetic sequence used in the production of the immunoglobulin idiotype. Hence, the idiotypic protein should serve as an ideal target for immune-based therapy of any B cell malignancy (or B cell clonal disease), if it can be produced in sufficient quantity.

To accomplish this, the Id protein of each patient must be identified and mass produced for use as an antigen in immunization. The identification may be accomplished by using the sequences of the framework region that are species-specific and PCR. Further, this process takes advantage of the relative clonal abundance of a B cell tumor. The tumor cells should be the most abundant population of antibody-producing cells in the body. Thus, techniques have been developed to isolate the portion of the gene required for immunotherapy. For one example, see PCT Application No. PCT/US01/25204, entitled “Method and Composition for Altering a B Cell Mediated Pathology” published on Feb. 21, 2002 as WO 02/13862, which is hereby incorporated by reference in its entirety including any drawings, figures and tables. A Notice of Allowance has been issued in corresponding U.S. application Ser. No. 09/927,121, and a U.S. Patent will issue. However, any suitable technique that allows all or part of the Id protein to be produced in sufficient quantities to be used for active immunotherapy can be used. Numerous techniques for this are available in the art.

This active immunotherapy avoids the phenomenon of mutational escape seen with passive immune strategies and has the potential to generate a broader immune response and thereby recognize the heterogeneous tumor cell population that can arise over time.

Following isolation of the gene, standard techniques can be used to produce the idiotype protein. A wide variety of techniques are available; in general, antibodies are produced in vitro using mammalian cells, yeast, bacterial, eukaryotic or prokaryotic cells. Mammalian cells produce functional antibody, including appropriate glycosylation. Bacterial cells are easier to grow and may be bioengineered to produce antibodies, however, the antibodies thus produced are not glycosylated. Other eukaryotic cells may be hosts for expression of endogenous genes and the antibodies produced are glycosylated. In the PCT publication, WO 02/13862, Gold and Shopes demonstrated that insect cells could be used to produce glycosylated idiotypic proteins, and further, that these idiotype proteins could be used to raise an immune response.

Induction of B Cell Apoptosis with an Anti-CD20 Antibody

As mentioned above, Rituxan® is a monoclonal antibody specific for the CD20 antibody found on mature B cells. Although its effect is specific for B cells, it is still attacks the entire population of a patient's B cells, rather than directing a specific anti-idiotype immune response directed specifically at malignant B cells.

If efficacy of immune stimulation following immunotherapy with an idiotype protein, as shown by Gold and Shopes, is due in whole or in large part to antibody production, then injection of idiotype protein immediately following treatment with Rituxan® would be a waste of resources; the therapy would be deprived of one arm of the immune system. Surprisingly, however, treatment with rituximab followed quickly by immunization with an Id protein produces a synergistic response: the depressed B cell population leads to an enhanced response to the Id protein immunization. This suggests to the inventors, without being bound by this or any other theory, that the Id protein therapy primarily functions by inducing, in vivo, a cell mediated (e.g., T cell) immune response for altering the B cell mediated pathology.

Chemotherapy Agents for the Treatment of B Cell Malignancies

Chemotherapeutic agents, as the term is herein, include a wide variety of small molecules such as DNA-interactive agents, antimetabolites, tubulin-interactive agents, hormonal agents and others, such as asparaginase or hydroxyurea. Chemotherapeutic agents may be selected from any of the following groups but are not limited thereto.

DNA-interactive agents include, but are not limited to, alkylating agents, such as cisplatin, cyclophosphamide, altretamine; DNA strand-breakage agents, such as bleomycin; intercalating topoisomerase II inhibitors, e.g., dactinomycin and doxorubicin); nonintercalating topoisomerase II inhibitors such as, etoposide and teniposide; and the DNA minor groove binder plicamydin.

Alkylating agents may form covalent chemical adducts with cellular DNA, RNA, or protein molecules, or with smaller amino acids, glutathione, or similar chemicals. Examples of typical alkylating agents include, but are not limited to, nitrogen mustards (bischloroethylamines), such as chlorambucil, cyclophosphamide, isofamide, mechlorethamine, melphalan, uracil mustard; aziridine such as thiotepa; methanesulphonate esters (or alkyl alkone sulfonates) such as busulfan; nitroso ureas, such as carmustine, lomustine, streptozocin; platinum complexes, such as cisplatin, carboplatin; and non classic alkylating agents such as procarbazine, dacarbazine and altretamine.

Antibiotic agents are a group of drugs produced in a manner similar to antibiotics as a modification of natural products. Examples of antibiotic agents include, but are not limited to, a bioreductive alkylator, such as mitomycin-C and DNA strand breaking agents that include bleomycin as an example. Other antibiotic agents include compounds that are thought to act as DNA topoisomerase II inhibitors and may include intercalators such as the following: amsacrine, dactinomycin, daunorubicin, doxorubicin (adriamycin), idarubicin, and mitoxantrone; as well as nonintercalators such as etoposide and teniposide. Other similar agents include epirubicin and anthracenedione.

Antimetabolites generally interfere with the production of nucleic acids and thereby growth of cells by one of two major mechanisms. Certain drugs inhibit production of deoxyribonucleoside triphosphates that are the precursors for DNA synthesis, thus inhibiting DNA replication. Examples of these compounds are analogues of purines or pyrimidines and are incorporated in anabolic nucleotide pathways. These analogues are then substituted into DNA or RNA instead of their normal counterparts.

Antimetabolites useful as chemotherapeutic agents include, but are not limited to, folate antagonists such as methotrexate and trimetrexate; pyrimidine antagonists, such as fluorouracil, fluorodeoxyuridine, CB3717, azacitidine, cytarabine, and floxuridine; purine antagonists such as mercaptopurine, 6-thioguanine, fludarabine, pentostatin; and ribonucleotide reductase inhibitors such as hydroxyurea. Other antimetabolites include pentostatin, fludarabine phosphate, cladribine (2-CDA), and gemcitabine.

Other agents include hydroxyurea (which appears to act primarily through inhibition of the enzyme ribonucleotide reductase), and asparaginase (an enzyme which converts asparagine to aspartic acid and thus inhibits protein synthesis).

Plant-derived agents are a group of drugs that are derived from plants or are modifications of the plant-derived agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel (Taxol) and docetaxel). Some of these plant-derived agents, such as some of the vinca alkaloids and paclitaxel, act as antimitotic agents by binding to tubulin and thereby disrupting mitosis. Podophyllotoxins such as etoposide are believed to interfere with DNA synthesis by interacting with topoisomerase II, leading to DNA strand scission. Another plant-derived chemotherapeutic agent is 20(S)-camptothecin.

Hormonal agents are also useful in the treatment of cancers and tumors and are indicated in hormonally susceptible tumors. Such agents are usually derived from natural sources. Hormonal agents include, but are not limited to, estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, and methyltestosterone. Adrenal corticosteroids are derived from natural adrenal cortisol or hydrocortisone and are used to treat B cell malignancies. They are used because of their anti-inflammatory benefits as well as the ability of some to inhibit mitotic divisions and to halt DNA synthesis. These compounds include, but are not limited to, prednisone, dexamethasone, methylprednisolone, and prednisolone.

Further chemotherapeutic agents include 2-CDA, 2′-deoxycofomycin, etoposide ifosfamide, and anthracycline.

A listing of some currently available chemotherapeutic agents according to class, and including diseases for which the agents are indicated, is provided as Table 3 of U.S. Pat. No. 6,608,096, which is hereby incorporated by reference in its entirety including any drawings, figures and tables.

The following combinations of chemotherapeutic agents, among others, are used to treat B cell and T cell malignancies: MOPP, ABVD, ChlVPP, CABS, MOPP plus ABVD, MOPP plus ABV, BCVPP, VABCD, ABDIC, CBVD, PCVP, CEP, EVA, MOPLACE, MIME, MINE, CEM, MTX-CHOP, EVAP and EPOCH. Other exemplary combinations of chemotherapeutic agents include, but are not limited to, CVP, CHOP, C-MOPP, CAP-BOP, m-BACOD, ProMACE-MOPP, ProMACE-CytaBOM, MACOP-B, IMVP-16, MIME, DHAP, ESHAP, CEPP(B), CAMP, CMP; COP, CAP, (vincristine, prednisone, anthracycline and cyclophosphamide or asparaginase); and (vincristine, prednisone, anthracycline, cyclophosphamide and asparaginase). For a detailed discussion of the chemotherapeutic agents and their method of administration, see Dorr, et al., Cancer Chemotherapy Handbook, 2d edition, Appleton & Lange (Connecticut, 1994) herein incorporated by reference. Further descriptions and protocols for chemotherapy regimes are well known to those skilled in the art.

Other agents used in cancer therapy include biotech agents that elicit cancer/tumor regression when used alone or in combination with chemotherapy and/or radiotherapy. Examples of biotech agents include, but are not limited to, immunomodulating proteins such as cytokines, monoclonal antibodies against tumor antigens, and compounds that will stimulate the immune system to attack the cancer cells.

All cytokines possess immunomodulatory activity. Some cytokines in particular, such as interleukin-2 (IL-2, aldesleukin) and interferon-α (IFN-α) have been approved for the treatment of patients with certain forms of cancer having demonstrated antitumor activity.

IL-2 is a T-cell growth factor that is central to T-cell-mediated immune responses. The selective antitumor effects of IL-2 on some patients may be the result of a cell-mediated immune response that discriminate between self and nonself. IFN-α has demonstrated activity against both solid and hematologic malignancies, and the later appear to be particularly sensitive. Other cytokines that useful in the instant invention include those cytokines that exert control on hematopoiesis and immune functions. Examples of such cytokines include, but are not limited to erythropoietin (epoietin-α), granulocyte-CSF (filgrastim), and granulocyte-macrophage-CSF (GM-CSF, sargramostim). Other biotech agents may be used in the instant invention provide a therapeutic effect against a B cell malignancy. Examples of such immunomodulating agents include, but are not limited to bacillus Calmette-Guerin, levamisole, and octreotide.

Without being bound to a particular theory, GM-CSF may be particularly useful in the present invention based on its ability to recruit dendritic cells to the injection site of autologous Id protein. Therefore, other agents that recruit dendritic cells to the injection site may also prove useful to the current invention.

Monoclonal Antibody Treatment

Monoclonal antibodies against antigens present on tumor cells are have been shown to be efficacious as therapeutic agents. In particular, monoclonal antibodies directed to the CD20 antigen (“an anti-CD20 antibody”) present on malignant B cells has proved efficacious in the treatment of B cell malignancies.

For example, the monoclonal antibody RITUXAN® (rituximab) was raised against CD20 antigen (also known as the Bp35 antigen) on lymphoma cells and selectively depletes normal and malignant CD20⁺pre-B and mature B cells. RITUXAN® is frequently used alone as a therapeutic agent for the treatment of patients with relapsed or refractory low-grade or follicular, CD20⁺, B cell non-Hodgkin's lymphoma. Another example of a monoclonal antibody used as a chemotherapeutic agent in cancer is HERCEPTIN™ (Trastruzuinab). HERCEPTIN™ was raised against human epidermal growth factor receptor2 (HER2) that was found to be over-expressed in some metastatic breast tumors. Over-expression of HER2 protein is associated with more aggressive disease and poorer prognosis in the clinic. HERCEPTIN™ is used as a treatment of patients with metastatic breast cancer whose tumors over-express the HER2 protein.

Rituxan® is approved for the treatment of patients with relapsed or refractory, low-grade or follicular, CD20-positive, B-cell non-Hodgkin's lymphoma. Results of the Phase III trial in 166 patients indicate an overall response rate of 48% (6% complete response (CR)), a median time to disease progression in all treated patients of 9.0 months and a median duration of response of 11.2 months [1.9 to 42.1+ months] (McLaughlin P, et al., “Rituximab chimeric anti-CD20 monoclonal antibody therapy for relapsed indolent lymphoma: half of patients respond to a four-dose treatment program,” J. Clin. Oncol. 1998; 16(8):2825-33)). The FDA has recently expanded the label to include re-treatment of patients who had achieved an objective response to a prior course of Rituxan®. In a multi-center, single-arm study, 60 patients received 375 mg/m² of Rituxan® weekly for 4 doses. (Davis T, et al. “Rituximab anti-CD20 monoclonal antibody therapy in non-Hodgkin's lymphoma: safety and efficacy of re-treatment,” J. Clin. Oncol. 2000; 18(17): 3135-43)). All patients had relapsed or refractory, low-grade or follicular B-cell NHL and had achieved an objective clinical response to a prior course of Rituxan®. Of these 60 patients, 55 received their second course of Rituxan®, 3 patients received their third course and 2 patients received their second and third courses of Rituxan® in this study. The over-all response rate (ORR) was 38% (10% CR and 28% partial response (PR)) with a projected median duration of response of 15 months (range, 3.0 to 25.1+ months). For vaccines which produce primarily an antibody response, there is a concern that combining immunotherapy with Rituxan®, which produces a rapid and sustained (up to 6 to 9 months post-treatment in 83% of patients) depletion of circulating and tissue-based B-cells, would blunt any antibody response.

Another anti-CD20 antibody, Bexxar® (tositumomab), has also recently been approved by the FDA for the treatment of B cell lymphomas (see e.g., U.S. Pat. Nos. 6,565,827; 6,287,537; 6,090,365; 6,015,542; 5,843,398; and 5,595,721). Other monoclonal antibody therapies in clinical trials or recently approved include Oncolym® (directed against the HLA-Dr10 protein), Zevalin®, epratuzumab, and an anti-CD52 antibody Campath®/MabCampath® (alemtuzumab).

Autologous Id Protein Immunotherapy

The present invention contemplates use of the unique antigen, the immunoglobulin idiotype (Id), on the B-cell malignancy as an immunotherapy. This Id contains protein sequences from both the variable immunoglobulin heavy and light regions (V_{H} and V_{L} ) set within the constant antibody domains.

By using the idiotype protein as an immunogen, the unique determinants that define the idiotype can be shown to instruct and activate the immune system to their existence leading to the subsequent recognition and destruction of the B cell malignancy from which the idiotype genes were originally cloned. The DNA sequence encoding the variable region of the idiotypic immunoglobulins is cloned using primers derived from the 5′ end of each unique subfamily of light and heavy immunoglobulin chains together with a constant region primer. Typically, this process uses one of several suitable cloning techniques such as PCR. These constant region primers, in combination with one for the V_{H} region and one for the V_{L} region, may be used to clone the variable regions as a first step in producing a chimeric protein comprising a variable region and a constant region. Alternatively, techniques such as 5′ RACE may be used. For the studies described herein, 5′ RACE was used to clone the variable regions of the heavy and light immunoglobulin chains in order to produce a chimeric protein. Examples of chimeric proteins include: V_{L}/Cλ, V_{L}/Cκ, V_{L}/IgG_{γ1}, V_{H}/IgG_{γ1}, V_{H}/Cλ, and V_{H}/Cκ. The chimeric protein thus comprises a portion of a variable region from an immunoglobulin molecule from a patient and also comprises a portion of a constant region from a source other than the patient. In preferred embodiments, the heavy and light chain constant region sequences are derived from 9F12 cells. However, other sources for immunoglobulin constant region genes may be used. These chimeric proteins are predicted to be more efficiently produced than using existing systems for producing idiotypic proteins and will be excellent immunogens for use in immunization protocols.

This means of inducing active immunotherapy may avoid the phenomenon of mutational escape seen with passive immune strategies. Such therapy has the potential to generate a broader immune response and thereby recognize the heterogeneous tumor cell population that can arise over time. The difficulty with active immunotherapy lies in convincing the patient's immune system to react against a perceived “self antigen” expressed by the tumor. Unlike the idiotypic protein found on B cell malignancies, many antigens expressed by tumors are weak immunogens.

To tailor the in vitro-produced idiotype protein to a particular patient first requires identification and isolation of the genes encoding the unique antigens, then the means of producing those antigens. This may be accomplished in a number of different ways available to one of skill in the art. For example, a recently developed method that is adapted to the needs of the instant invention uses a novel baculovirus/insect cell expression system and was recently developed for the efficient production of functional antibodies for immunotherapy (see, for example, PCT Application No. PCT/US01/25203, entitled “Method and Composition for Altering a T Cell Mediated Pathology” published as WO 02/13861 on Feb. 21, 2002, which is hereby incorporated by reference in its entirety including any drawings, figures and tables).

In the invention described in WO 02/13861 and WO 02/13862, these chimeric proteins are produced in insect cells using a baculovirus vector. The baculovirus/insect cell expression system employed has been proven effective as shown by the efficient production of functional antibodies and Id proteins for immunotherapy from numerous patients. Expression of recombinant proteins using the baculovirus system allows the production of large quantities of biologically active proteins without many of the drawbacks associated with proteins made in bacteria, and also avoids the complications of using mammalian cells. For example the immunoglobulin genes from the stable human cell-line 9F12 (ATCC#HB8177), which produces a human IgG_{γ1}/κ antibody specific for tetanus toxoid, were cloned into a baculovirus dual promoter expression transfer vector. Intact IgG_{γ1}/κ immunoglobulin was produced in insect cells that behaved similarly to the mammalian antibody in SDS-PAGE analysis and Western blots. The antibody produced by insect cells was glycosylated. The binding affinities of purified Mab9F12 and purified baculovirus expressed antibody were determined to be identical and production levels were determined to be approximately 5-10 μg/ml.

Thus, the baculovirus expression system is an excellent alternative to antibody production in E. coli and mammalian cells. High yield production (1-100 mg/L) of biologically active proteins in eukaryotic cells is possible using the baculovirus system (Haseman et al., Proc. Natl. Acad. Sci. 87:3942-46, 1990). The baculovirus/insect cell system circumvents the solubility problems often encountered when recombinant proteins are over-expressed in prokaryotes. In addition, insect cells contain the post translational modification machinery responsible for correct folding, disulfide formation, glycosylation, P-hydroxylation, fatty acid acylation, prenylation, phosphorylation and amidation of eukaryotic cells, but not present in prokaryotes. The production of a functional, glycosylated monoclonal antibody recognizing human colorectal carcinoma cells from a baculovirus expression system has been demonstrated (Nesbit, J. et al., Immunol. Methods, 151:201-208, 1992). This baculovirus-produced antibody was shown to mediate ADCC; in contrast, antibodies produced in bacteria are not glycosylated and hence have no detectable ADCC activity. However, the glycosylation produced by insect cells recombinant protein production is not identical to that attached when recombinant proteins are produced in mammalian cells. (See, e.g., Altmann et al., Glycoconjugate J 16:109-123 (1999).

Methods

Production of Autologous Id Variable Proteins

A composition is administered that contains at least one chimeric protein having at least a portion of a V_{H} or V_{L} region of an immunoglobulin variable region derived from a patient and at least a portion of an immunoglobulin constant region. The V_{H} or V_{L} region used in this composition is associated with a particular immunoglobulin produced by a B cell from a patient having a B cell mediated pathology. The composition may contain two different chimeric proteins. Each chimeric protein has at least a portion of a V_{H} and/or V_{L} region of an immunoglobulin chain derived from a patient linked to at least a portion of an immunoglobulin constant region. The V_{H} and/or V_{L} regions that are part of the chimeric protein are associated with particular immunoglobulin chains from a B cell of the patient having a B cell mediated pathology. Thus, specific immunoglobulin chains containing patient-derived unique V_{H} and/or V_{L} chains can be developed as therapeutic compositions. The Id protein administered to the patient may also be the entire V_{H}C_{H} plus V_{L}C_{L} as derived from the patient, or any portion thereof.

The immunoglobulin constant regions used in the above compositions and chimeric protein can be from IgG₁, IgG₂, IgG₃, IgG₄ IgA₁, IgA₂, IgM, IgD, IgE heavy chains, and κ or λ light chains or portions thereof. In some of the embodiments, the chimeric protein only contains either the V_{H} and/or V_{L} region of an immunoglobulin region with an immunoglobulin constant region. Examples of chimeric proteins include V_{H}-IgG_{λ1}, V_{L}-κ, and V_{L}-λ. In another embodiment, the composition contains two chimeric proteins that each respectively contains a V_{H} and V_{L} region with an immunoglobulin constant region. Examples include V_{H}-IgG_{γ1} and V_{L-}κ, and V_{H}-IgG_{65 1} and V_{L}-λ.

A chimeric protein may be produced using recombinant DNA technology and an expression system. This method includes the following steps: (a) isolating genes encoding the V_{H} or V_{L} region of an immunoglobulin chain from B cells of a patient having a B cell mediated pathology, (b) inserting the isolated gene encoding the V_{H} or V_{L} region of an immunoglobulin chain and the gene encoding an immunoglobulin constant region into an expression vector to allow the expression of a chimeric protein, (c) producing the chimeric protein by introducing the expression vector into cells and allowing its expression, and (d) isolating the chimeric protein. The method for producing chimeric proteins further includes a step of inserting a gene encoding either the V_{H} and/or V_{L} region of an immunoglobulin chain and a gene encoding a second immunoglobulin constant region into the expression vector to allow the expression of the second chimeric protein.

The first of the chimeric proteins may comprise the entire V_{H} region and a human constant region of an immunoglobulin IgGγ1 (V_{H}-IgG_{γ1}), and the second chimeric protein may comprise the entire V_{L} and a human κ or λ constant region (V_{L}-C_{κ} V_{L}-C_{λ}). Either or both of the chimeric proteins may comprise at least a portion of a V_{H} and/or V_{L} region of an immunoglobulin chain, plus a linker region, and at least a portion of an immunoglobulin constant region.

The composition may contain a single chimeric protein containing either a V_{H} and/or V_{L} region from a particular immunoglobulin chain from a B cell of a patient and an immunoglobulin constant region. Examples include chimeric proteins V_{H}-IgG_{γ1} , V_{L}-κ, V_{L}-λ, V_{L}-IgG_{γ1}, V_{H}-κ, and V_{H}-λ).

The expression vector used to express the chimeric proteins may be a baculovirus expression vector. The chimeric proteins may be expressed in insect cells. The expression vector used to express the chimeric proteins may be a yeast expression vector. The chimeric proteins may be expressed in yeast cells. The expression vector used to express the chimeric proteins may be a mammalian cell expression vector. The chimeric proteins may be expressed in mammalian cells. The expression vector used to express the chimeric proteins may also be a bacterial expression vector. The chimeric proteins may be expressed in bacterial cells.

A vector for use in the methods of the invention preferably contains two expression cassettes each having a promoter, a secretory signal sequence and a chimeric protein. In one embodiment, the expression cassette may contain the baculovirus AcNPV p10 promotor linked to the honey bee melittin secretory signal sequence. The other expression cassette may have the polyhedrin promotor linked to a human placental alkaline phosphatase secretory signal sequence. Other promoters and signal sequences may also be used. In some preferred embodiments, the signal sequences are endogenous signal sequences associated with the V_{H} and V_{L} genes isolated from patients, or other signal sequences involved in antibody production. In the baculovirus expression embodiment, the genes encoding the V_{H} or V_{L} portions of the immunoglobulin chains, and the genes encoding immunoglobulin constant region are inserted, separately and/or together, into the above expression cassette of the baculovirus vector allowing expression of one or two chimeric proteins. In one preferred embodiment, the constant region of the immunoglobulin heavy chain, such as IgG_{γ1}, with either the V_{H} or V_{L} region, is controlled by the polyhedrin promotor. The polyhedrin promoter may be operatively linked to either the honey bee melittin promoter or the human alkaline phosphatase promoter, as may the p10 promoter.

Chimeric proteins produced are purified using affinity columns with anti-immunoglobulin antibodies or Ig-binding proteins, such as protein A for the constant region of an immunoglobulin heavy chain, and protein L for κ light chains, and/or any other proteins that bind to an immunoglobulin binding domain.

The chimeric proteins may be covalently coupled to a carrier protein such as a keyhole limpet hemocyanin (KLH). The composition of may also be administered into a patient together with a cytokine such as granulocyte-macrophage-CSF (GM-CSF), or a chemokine such as a monocyte chemotactic protein 3 (MCP 3). Because the present containing chimeric protein(s) is specifically related to a particular immunoglobulin from B cells of a patient having B cell mediated pathology, administration of this composition induces an immune response against the disease specific idiotype in which particular V_{H} or V_{L} segments are involved. The administration routes for the invented composition include but are not limited to oral delivery, inhalation delivery, injection delivery, transdermal delivery, and the like.

This technique takes advantage of the unique cell surface antigens present on the surface of B cells involved in B cell pathologies, and are prepared in a patient-specific manner. Such autologous Id proteins provide exquisite selectivity in directing a patient's immune system to the unique markers associated with the pathogenic B cells found in a given patient.

Baculovirus Expression of Recombinant Proteins

As discussed supra, the expression of recombinant proteins using the baculovirus system has emerged as an excellent choice for high yield production (1-100 mg/L) of biologically active proteins in eukaryotic cells. Nesbit and co-workers demonstrated the production of a functional, glycosylated monoclonal antibody from a baculovirus expression system (Nesbit, J., et al., Immunol. Methods 151:201-208, 1992). Additionally, expression of recombinant IgA has also been demonstrated in baculovirus cells, and this IgA was correctly assembled into heavy chain/light chain heterodimers, N-glycosylated, and secreted (Carayannopoulos et al., Proc. Natl. Acad. Sci. 91:8348-52, 1994, PCT Publication No. WO 98/30577, U.S. Pat. No. 6,063,905).

The baculovirus/insect cell expression system has proven effective for the efficient production of functional antibodies and Id proteins for immunotherapy from any given patient. One baculovirus expression vector as described in WO 02/13862 was designed such that only two custom gene-specific primers are needed to amplify any pair of antibody variable regions for easy subcloning and expression as chimeric proteins based on the human κ light chain and IgG_{γ1} heavy chain. The incorporation of heterologous secretary signal sequences, which direct the heavy and light chains to the secretary pathway of insect cells, were incorporated for the expression of large amounts of active immunoglobulin. This vector is useful for the expression of any κ light chain variable region (V_{L}) in frame with human κ constant region and secreted via the human placental alkaline phosphatase secretary signal sequence; and any heavy chain variable region (V_{H}) in frame with the human IgG_{γ1} constant domain led by the honey bee melittin secretary signal sequence. The λ light chain constant region may replace the κ constant region. The chimeric protein is then expressed with the V_{L} region in frame with human λ constant region and secreted via the human placental alkaline phosphatase secretary signal sequence, along with any heavy chain variable region (V_{H}) in frame with the human IgG_{γ1} constant domain led by the honey bee melittin secretary signal sequence. Any monoclonal antibody, mouse or human, either from a monoclonal cell line or identified by phage display cloning, could be easily expressed as whole human IgG_{γ1}/λ or IgG_{γ1}/κ in this vector after two simple subcloning steps. Additionally, different immunoglobulin types, including IgG_{γ2}, IgG_{γ3}, IgG_{γ4}, IgA, IgA, IgA₁, IgA₂, IgM, IgD, IgE heavy chains, or segments thereof, could be used in place of the IgG_{γ1} constant region. Furthermore, besides those signal sequences described supra, the instant invention may use other secretory signal sequences such as the endogenous secretory sequences associated with the immunoglobulin genes derived from a given patient. Additionally, one of skill in the art would be able to select several different primers that could be used equivalently in this system to produce equivalent results to amplify any pair of antibody variable regions for easy subcloning.

In some instances, utilization of the baculovirus system for the expression of biologically active proteins has been hampered by the inability to efficiently solubilize recombinant proteins without excessive proteolytic degradation. In order to circumvent solubility and proteolysis problems encountered with the expression of recombinant proteins in insect cells, baculovirus transfer vectors have been developed for the efficient secretion of biologically active proteins. These vectors that facilitate the secretion of recombinant proteins from host insect cells are constructed by inserting functional secretory leader sequences downstream of the polyhedrin promoter, p10 promoter, or other promoters. In-frame insertion of cDNA sequences resulted in the synthesis of proteins containing a heterologous signal sequence which directed the recombinant protein to the secretory pathway. Other human and insect leader sequences may be tested to maximize secretion of heterologous proteins from insect cells. For examples of leader sequences, see the WO 02/13862 publication by Gold and Shopes.

Additionally, baculovirus vectors can be engineered to express large amounts of the protein of interest in cultured insect cells (e.g., Sf9 cells) (Jasny, Science 238:1653, 1987; Miller et al., in: Genetic Engineering, Vol. 8, Plenum, Setlow et al., eds., pp. 277-297, 1986). Vectors which may be used include the pAc series (Smith et al. (1983) Mol. Cell Biol. 3:2156-2165) and the pVL series (Lucklow and Summers (1989) Virology 170:31-39).

Other Expression Systems for Recombinant Proteins

Proteins, protein fragments, and analogs may also be synthesized, in whole or in part, by recombinant methods using expression vectors encoding all or part of a protein.

Prokaryotic hosts

Prokaryotic hosts are, in generally, very efficient and convenient for the production of recombinant polypeptides and are, therefore, one type of preferred expression system. Prokaryotes most frequently are represented by various strains of E. coli, but other microbial strains may be used, including other bacterial strains. Recognized prokaryotic hosts include bacteria such as E. coli, Bacillus, Streptomyces, Pseudomonas, Salmonella, Serratia, and the like. Genetic engineering of prokaryotic hosts is possible so that these hosts will glycosylate proteins. (See, e.g., Wacker et al., N-linked glycosylation in Campylobacter jejuni and its functional transfer into E. coli” Science (2002) 298(5599):1790-93).

In prokaryotic systems, vectors that contain replication sites and control sequences derived from a species compatible with the host may be used. Preferred prokaryotic vectors include plasmids such as those capable of replication in E. coli (such as, for example, pBR322, ColE1, pSC101, pACYC 184, VX, pUC118, pUC119 and the like). Suitable phage or bacteriophage vectors may include λgt10, λgt11, vectors derived from filamentous bacteriophage such as m13, and the like. Suitable Streptomyces plasmids include p1J101, and Streptomyces bacteriophages such as phi.C31. Bacillus plasmids include pC194, pC221, pT127, and the like. Suitable Pseudomonas plasmids have been reviewed by Izaki (Jpn. J. Bacteriol. 33:729-742, 1978) and John, et al., (Rev. Infect. Dis. 8:693-704, 1986).

To express a protein (or a functional derivative thereof) in a prokaryotic cell, it is necessary to operably link the sequence encoding the protease of the invention to a functional prokaryotic promoter. Such promoters are either constitutive or inducible promoters, but commonly inducible promoters are used. Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ, the trp, recA, lacZ, lacI, and gal promoters of E. coli, the α-amylase and the sigma-28-specific promoters of B. subtilis, the promoters of the bacteriophages of Bacillus, and Streptomyces promoters. Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984). Additionally, proper expression in a prokaryotic cell also requires the presence of a ribosome-binding site upstream of the gene sequence-encoding sequence. Such ribosome-binding sites are disclosed, for example, by Gold, et al., (Ann. Rev. Microbiol. 35:365-404, 1981).

Fusion Proteins

Proteins may be expressed as fusion proteins. Genes for proteins expressed as fusion proteins ligated into expression vectors that add a number of amino acids to a protein encoded and expressed, usually to the amino terminus of the recombinant protein. Such a strategy of producing fusion proteins is usually adopted for three purposes: (1) to assist in the purification by acting as a ligand in affinity purification, (2) to increase the solubility of the product, and (3) to increase the expression of the product. Often, expression vectors for use in fusion protein production, a proteolytic cleavage site is included at the junction of the fusion region and the protein of interest to enable purification of the recombinant protein away from the fusion region following affinity purification of the fusion protein. Such enzymes, and their cognate recognition sequences, include Factor Xa, thrombin and enterokinase, and may also include trypsin or chymotrypsin. Typical fusion expression vectors include pGEX (Pharmacia Biotech Inc; Smith, D. B. and Johnson, K. S., Gene 67:31-40, 1988), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Improving Yield

Maximizing recombinant protein expression in E. coli can be assisted by expressing the protein or fusion protein in a host bacteria with an impaired proteolytic system so as to reduce the post-synthesis degradation of the recombinant protein (Gottesman, S., Gene Expression Technology: Methods in Enzymology 185, Academic Press, San Diego, Calif. 119-128, 1990). Another strategy is to alter the mix of codons used in the coding sequence to reflect the usage of the individual codons for each amino acid in the host (e.g., E. coli (Wada, et al., Nucleic Acids Res. 20:2111-2118, 1992). Such alteration of nucleic acid sequences of the invention can be carried out by standard DNA synthesis techniques and may prove useful for a variety of prokaryotic and eukaryotic expression systems.

Eukaryotic Hosts

Suitable hosts may include eukaryotic cells. Preferred eukaryotic hosts include, for example, yeast, fungi, insect cells, and mammalian cells both in vivo and in tissue culture. Useful mammalian cell hosts include HeLa cells, cells of fibroblast origin such as VERO or CHO-K1, and cells of lymphoid origin and their derivatives, as well as the NSO myeloma cell line. Preferred mammalian host cells include SP2/0 and J558L, as well as neuroblastoma cell lines such as IMR 332, which may provide better capacities for correct post-translational processing. Other preferred cell lines that may be used for the expression of recombinant genes include other variants of CHO cells, mouse L cells, BW5147 cells, and variants thereof. In general, eukaryotic organisms such as yeast provide substantial advantages in that they can also carry out post-translational modifications.

A large number of yeast expression systems may be potentially utilized which incorporate promoter and termination elements from the actively expressed sequences coding for glycolytic enzymes. These expression systems produce large quantities of proteins when yeast are grown in mediums rich in glucose. Known glycolytic gene sequences can also provide very efficient transcriptional control signals. A number of recombinant DNA strategies exist utilizing strong promoter sequences and high copy number plasmids which can be utilized for production of the desired proteins in yeast. Examples of vectors suitable for expression in S. cerevisiaeinclude pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.). Expression vectors incorporating MFα (α-factor) signal and leader peptides have been frequently used for expression of heterologous proteins in yeast; for example, see U.S. Pat. Nos. 5,162,498 and 4,546,082. In the '082 patent, a gene of interest was fused to the S. cerevisiae MFα signal/leader sequence to provide for secretion and processing of the protein of interest. Another yeast expression system is described in U.S. Pat. No. 6,183,989. Additionally, Pichia pastoris may be used to express heterologous proteins (Cregg, et al., “Recombinant protein expression in Pichia pastoris,” Mol. Biotechnol. 16:23 (2000)). Further, new techniques have been developed to express proteins in yeast with a more human-like glycosylation pattern (Hamilton, R., et al., Science 301:1244-46 (2003)).

In another embodiment, the protein of interest may be expressed in insect cells, for example the Drosophila larvae, but also including the methods discussed supra. Using insect cells as hosts, the Drosophila alcohol dehydrogenase promoter may be used (Rubin, Science 240:1453-1459, 1988). Sf9 cells may also be used, together with vectors such as the pAc series (Smith et al., Mol. Cell Biol. 3:2156-65 (1983)), or the pVL series (Lucklow et al., Virology 170:31-39 (1989)).

Plant cells may also be utilized as hosts, and control sequences compatible with plant cells are available, such as the cauliflower mosaic virus 35S and 19S promoters, and nopaline synthase promoter and polyadenylation signal sequences. Furthermore, the protein of interest may be expressed in plants which have incorporated the expression vector into their germ line.

Mammalian Expression Vectors

In yet another embodiment, a nucleic acid of the invention may be expressed in mammalian cells using a mammalian expression vector. Techniques for expression in mammalian cells have recently been summarized (Colosimo, et al., “Transfer and expression of foreign genes in mammalian cells,” Biotechniques 29(2):314-8, 320-2, 324 passim, 2000; which is hereby incorporated by reference in its entirety including any drawings, tables, and figures.). Examples of mammalian expression vectors include pCDM8 (Seed, B. (1987) Nature 329:840) and pMT2PC (Kaufmnan et al. (1987) EMBO J. 6:187-193). For use in mammalian cells, the regulatory sequences of the expression vector are often derived from viral regulatory elements. For example, commonly used promoters are derived from Simian Virus 40 (SV40), polyoma, Adenovirus 2, and cytomegalovirus (CMV) viruses. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast ga14 gene sequence promoter (Johnston et al., Proc. Natl. Acad. Sci. (USA) 79:6971-6975, 1982; Silver et al., Proc. Natl. Acad. Sci. (USA) 81:5951-5955, 1984). Alternatively, promoters from mammalian expression products, such as actin, collagen, myosin, and the like, may be employed. Regulatory elements may also be derived from adenovirus, bovine papilloma virus, cytomegalovirus, simian virus, or the like.

Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation. Expression of proteins of interest in eukaryotic hosts requires the use of eukaryotic regulatory regions. Such regions will, in general, include a promoter region sufficient to direct the initiation of RNA synthesis.

The recombinant mammalian expression vector may also be designed to be capable of directing expression of the nucleic acid preferentially in a particular cell type (i.e., tissue-specific regulatory elements are used to control the expression). Such tissue-specific promoters include the liver-specific albumin promoter (Pinkert et al. (1987) Genes Dev. 1:268-277); lymphoid-specific promoters (e.g., Calame and Eaton (1988) Adv. Immunol. 43:235-275), and in particular promoters of immunoglobulins and T cell receptors ((Winoto and Baltimore (1989) EMBO J. 8:729-733, Banerji et al. (1983) Cell 33:729-740; Queen and Baltimore (1983) Cell 33:741-748); mammary gland-specific promoters (e.g., milk whey promoter; U.S. Pat. No. 4,873,316 and European Application Publication No. 264,166); and pancreas-specific promoters (Edlund et al. (1985) Science 230:912-916). Developmentally-regulated promoters may also be utilized, for example, the α-fetoprotein promoter (Camper and Tilghman (1989) Genes Dev. 3:537-546), and the murine hox promoters (Kessel and Gruss (1990) Science 249:374-379).

Preferred eukaryotic plasmids include, for example, SV40, BPV, pMAM-neo, pKRC, vaccinia, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance,” Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, DEAE-dextran-mediated transfection, lipofection, calcium phosphate-precipitation, direct microinjection, and the like. Suitable methods for transforming or transfecting host cells can be found in Sambrook, et al. (“Molecular Cloning: A Laboratory Manual,” Third edition, Cold Spring Harbor Laboratory, 2001). After the introduction of the vector, recipient cells are grown in a selective medium, which selects for the growth of vector-containing cells. Expression of the cloned gene(s) results in the production of a protein of interest, or fragments thereof.

Other suitable expression systems for both prokaryotic and eukaryotic cells are well known to one skilled in the art and further are described in laboratory manuals such as Sambrook et al., 2001 or Ausubel et al. (“Current Protocols in Molecular Biology,” John Wiley & Sons, 1998) both of which are incorporated herein by reference in their entirety, including any drawings, figures or tables.

For transformation of eukaryotic cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, neomycin, methotrexate, glyphosate, and bialophos. Nucleic acid encoding a selectable marker can be introduced into a host cell on the same vector as that encoding the protein of interest or can be introduced on a separate vector. Cells stably transformed with the introduced nucleic acid can be identified by drug selection (e.g., cells that have incorporated the selectable marker gene will survive, while the other cells die).

In other embodiments, the DNA gene sequence used for expression of the recombinant product may be adjusted for the preference of the host organism. Further, the codon preference may also be adjusted for the tissue of origin of the host cell (see, e.g., Plotkin J B, et la., “Tissue-specific codon usage and the expression of human genes,” Proc Natl Acad Sci USA. Aug. 24, 2004; 101(34):12588-91).

A host cell of the invention, such as a prokaryotic or eukaryotic host cell in culture, can be used to produce (i.e., express) the protein of interest. Accordingly, the invention further provides methods for producing the protein of interest using the host cells of the invention. In one embodiment, the method comprises culturing the host cell into which a recombinant expression vector encoding the protein of interest has been introduced in a suitable medium such that the protein of interest is produced, and may be purified by one skilled in the art.

Vectors, or preferably expression vectors, may contain a gene encoding a polypeptide of interest such as an idiotype protein. These vectors may be employed to express the encoded polypeptide in either prokaryotic or eukaryotic cells.

In Vitro Transcription and Translation

Proteins, protein fragments, and analogs may also be synthesized using recombinant assembly of an expression vector followed by in vitro transcription and translation.

DNA Vaccine

The patient's own cells can produce proteins and protein fragments when provided with a DNA vaccine. A recombinant plasmid can be constructed encoding the gene of interest and then used as a DNA vaccine.

Treatment of the Patient

The compositions used in the present invention preferably are sterile and contain a therapeutically effective amount of an active substance for producing the desired response in a weight or volume suitable for administration to a patient. Factors within the knowledge and expertise of the health practitioner administering the composition include: the particular condition being treated, the severity of the condition, the patient's age, the patient's physical condition, the patient's weight, the duration of the treatment, the nature of concurrent therapy (if any), the route of administration and like factors. These factors are well known to those of ordinary skill in the art and can be addressed with no more than routine adjustments. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for other reasons.

The response of the patient can be measured by assays well known to one of ordinary skill in the art. Such assays can be employed for measuring the level of the response.

Other protocols for the administration of therapeutic compositions will be known to one of ordinary skill in the art, in which the dose amount, schedule of injections, sites of injections, mode of administration and the like vary from the foregoing. Administration of therapeutic compositions to mammals other than humans, e.g. for testing purposes or veterinary therapeutic purposes, is carried out under substantially the same conditions as described above.

### EXAMPLES

### Example 1

### Treatment of a B-Cell NHL Patient Relapsed/Refractory from Prior Lymphoma Therapies Using a Specific Binding Cytoreductive Agent and Autologous Id Protein

Study Rationale

Rituxan® is approved for the treatment of patients with relapsed or refractory, low-grade or follicular, CD20-positive, B-cell non-Hodgkin's lymphoma (See, e.g., McLaughlin P, et al., “Rituximab chimeric anti-CD20 monoclonal antibody therapy for relapsed indolent lymphoma: half of patients respond to a four-dose treatment program,” J Clin Oncol. (1998) 16(8):2825-33). Its use in conjunction with immunizations of Id-KLH plus GM-CSF was studied as set forth below. In mouse model systems, there is evidence that for immunizations that induce strong T-cell responses, such as Id-KLH plus GM-CSF, depleting the host of B-cells could actually increase the T-cell response to the immunogen. (Qin Z, et al., “B cells inhibit induction of T cell-dependent tumor immunity,” Nature Med. 4(5): 627-30(1998); Monach P A, et al., “CD4+ and B lymphocytes in transplant immunity. II. Augmented rejection of tumor allografts by mice lacking B cells,” Transplantation 55(6): 1356-61 (1993); Epstein M M, et al., “Successful T-cell priming in B-cell deficient mice,” J. Exp. Med. 182(4): 915-22 (1995)).

The NCI is currently conducting a Phase 2 trial of Id-KLH plus GM-CSF immunotherapy following chemotherapy and Rituxan® in patients with mantle cell lymphoma. They are evaluating the effect of Rituxan® on the ability of patients to mount a T-cell response to their idiotype immunogen. In addition, recent pre-clinical studies in a T-cell lymphoma murine model were conducted at Favrille, Inc., utilizing genetically B-cell deficient mice. In these studies, animals that were treated demonstrate an enhanced protective response to Id-KLH in combination with GM-CSF. In these studies, immunized mice developed significantly fewer tumors compared to KLH/GM-CSF treated controls. In light of these observations, the study below evaluated the combination of FavId™ active immunization with Rituxan®. Patients in this trial who have relapsed or refractory grade 1 or 2 follicular lymphoma following chemotherapy or who have relapsed following Rituxan®, were eligible to receive Rituxan® plus FavId™ and GM-CSF.

This protocol was to evaluate the ability of patients treated with Rituxan® plus FavId™ and GM-CSF to mount an immune response (humoral and/or cellular) to KLH and their idiotype. Additionally the objective response rate, median duration of response and median time to progression produced by Favld and GM-CSF in combination with Rituxan® was evaluated.

Study Design

(1) General

This was an open-label, single-arm Phase II study designed to evaluate whether patients treated with Rituxan® can mount an immunologic response to FavId™ plus GM-CSF following shortly after rituximab treatment.

All patients had a lymph node biopsy to obtain tissue for morphological classification, immunophenotypic characterization and to provide material for generation of FavId™. Only patients with Grade 1 or 2 follicular lymphoma whose biopsies were found to express monoclonal surface immunoglobulins (indicative that tumor has been isolated) were accepted as study candidates. Remaining biopsy cell suspensions were stored in liquid nitrogen for archival purposes. Some of the material, however, may be used for in vitro immunological response studies to detect the presence of tumor specific antibodies in the sera of treated patients or of tumor reactive T lymphocytes from the peripheral blood of treated patients. In addition, DNA may be prepared from remaining tissue samples to establish the presence of a t(14;18) translocation in the tumor preparation. Should such a translocation be identified, subsequent assessment of the occurrence of this translocation in peripheral blood lymphocytes may be performed. If greater than 18 months elapses between biopsy and therapy administration, or if a patient begins to progress following an initial response, a second biopsy, core biopsy, or fine-needle aspirate may be obtained to confirm molecular characteristics of the therapeutic product or to determine genetic similarity to initial tumor in patients with tumor progression.

For entry, patients must have at least one remaining bi-dimensionally measurable lesion measuring at least 2 cm in each dimension. CT scans of all lymph nodes was obtained at baseline (as close to Rituxan® administration as possible but within 1 month in any case). Repeat CT scans were obtained every 3 months thereafter. A second original were obtained and submitted to Favrille for central blinded review.

Eligible patients received 4 weekly infusions of Rituxan® at a dose of 375 mg/m² followed approximately 8 weeks later by 6 monthly subcutaneous FavId™/GM-CSF treatments. Patients with an objective response (CR or PR) or lack of progressive disease could continue to receive FavId™/GM-CSF treatments every other month for 6 treatments and then every 3 months provided there is/was no evidence of disease progression. All patients were monitored closely for toxicity throughout the study. Patients had serial monitoring of immune response (humoral and T-cell responses). Serum anti-idiotypic and anti-KLH antibody were assessed from samples obtained at baseline (prior to Rituxan®) and before each FavId™ treatment and at 4 weeks following the last FavId™ treatment. Idiotype-specific and KLH-specific proliferation of PBMC were also be measured from samples obtained at baseline (prior to Rituxan®) and before each FavId™ treatment and four weeks following the completion of FavId™ therapy.

Drug Administration

(1) Rituxan®

Rituxan® was supplied by each study site and was administered as an intravenous infusion according to the package insert once a week for 4 weeks. Prescribing details as supplied by the manufacturer were used.

(2) FavId™ and GM-CSF

FavId™ and GM-CSF were supplied by Favrille, Inc. and began approximately 8 weeks following the completion of Rituxan® (as soon as permitted by the completion of the production of FavId™). FavId™ was produced in part according to the methods set forth in PCT Application No. PCT/US01/25204, entitled “Method and Composition for Altering a B Cell Mediated Pathology” published on Feb. 21, 2002 as WO 02/13862.

FavId™, 1 mg (0.5 mg of tumor Id protein conjugated to 0.5 mg of KLH carrier protein) and 250 mcg of soluble GM-CSF were selected based on prior clinical experience with similar treatment schedules and doses. (See, e.g., Hsu F J, et al., “Tumor-specific idiotype vaccines in the treatment of patients with B- cell lymphoma—long-term results of a clinical trial,” Blood 89(9):3129-35 (1997); Bendandi M, et al., “Complete molecular remissions induced by patient-specific vaccination plus granulocyte-monocyte colony-stimulating factor against lymphoma,” Nat. Med. 5(10):1171-7 (1999); Massaia M, et al., “Idiotype vaccination in human myeloma: generation of tumor-specific immune responses after high-dose chemotherapy,” Blood 94(2):673-83 (1999)). Patients received a subcutaneous injection of FavId™ with GM-CSF (split into two injection sites) once a month for six months. A flat dose of 250 mcg of GM-CSF alone was administered by subcutaneous injection for 3 days following each FavId™ injection. All GM-CSF injections were also split (125 mcg per injection site) and given in close proximity to the FavId™ injection sites, as close to the exact sites of the FavId™ Day 1 injection sites as possible. The injection sites were rotated between the upper and lower extremities each month.

The administration of GM-CSF alone was not necessarily performed at the principal investigator's site but in some cases might have been self-administered by the patient or their designee following appropriate instruction.

Possible GM-CSF toxicity was monitored, and GM-CSF could be adjusted as appropriate. Patients were observed for adverse events for 1 hour following FavId™ and GM-CSF Day 1 administration of each course of therapy.

(3) Outline of Study Procedures/Schedule of Assessments

Patients were treated according to the schedule outlined below. Following completion of 4 weekly treatments with Rituxan®, patients received subcutaneous injections of FavId plus GM-CSF over the course of 6 months unless there was documented unmanageable toxicity or clinically significant disease progression requiring intervention with other anti-cancer therapy. Patients who achieved an objective response (CR or PR) or lack of progressive disease (PD) might have continued to receive FavId™/GM-CSF treatments every other month for 6 treatments and then every 3 months provided there was no evidence of disease progression. Assessment of tumor response was obtained by CT scan at three month intervals thereafter. Safety assessments of all patients was made at least monthly during study treatment and at three month intervals thereafter while on treatment.

Study Population

(1) Inclusion Criteria

Patients were at least 18 years of age. Patients had either relapsed or were refractory following chemotherapy or relapsed following Rituxan® treatment. (Note: Rituxan® may have been given as second-line therapy following an initial response to chemotherapy or in combination with chemotherapy for initial therapy of their disease.) Patients in the study had a tumor accessible for biopsy or previous recent biopsy material was available; patients also had at least one additional bidimensional lesion measuring at least 2 cm in each dimension. They were suffering from histologically confirmed grade 1 or 2 follicular B-cell lymphoma (WHO classification), with a performance status (ECOG) of 0, 1 or 2; absolute granulocyte count greater than or equal to 1,000/mm³ (patients with greater than 5,000 lymphocytes were excluded); platelets greater than 100,000/mm³; total bilirubin less than or equal to 2 mg/dL; AST and ALT less than or equal to 2× upper limit of normal, and creatinine less than or equal to 1.5 mg/dL.

Patients were excluded from the study if (1) they were refractory to Rituxan® or chemotherapy or chemotherapy plus Rituxan®; (2) they had more than two prior treatment regimens (e.g., CHOP plus Rituxan® is one treatment regimen; CHOP followed by Rituxan® at initial relapse equals two treatment regimens); (3) prior fludarabine less than one year before start of FavId™ treatment; (4) patients with greater than 5,000 lymphocytes; (5) they had prior tumor-specific idiotype immunotherapy; (6) they were undergoing concurrent immunosuppressive therapy (high-dose steroids, etc.); (7) they had a prior splenectomy; (8) they had a known history of CNS lymphoma or meningeal lymphomatosis; (9) they were HIV positive; (10) they had serious non-malignant disease (e.g., psychiatric disorders, compromised pulmonary function (e.g. active asthma, COPD, pneumonitis, bronchiolitis obliterans), congestive heart failure, or active uncontrolled bacterial, viral, or fungal infections), or other conditions which, in the opinion of the investigator would have compromised protocol objectives; (11) prior malignancy (excluding nonmelanoma carcinomas of the skin and in situ cervical carcinomas) unless in remission for greater than or equal 2 years; (12) treatment with an investigational drug within 30 days prior to study entry; or (13) pregnant or nursing women (women of childbearing potential were advised to avoid becoming pregnant while receiving study treatment).

Patient Enrollment

Patients who were ready to be enrolled in the study were evaluated for eligibility by the investigator. Patients who met the eligibility criteria were enrolled prior to biopsy and assigned a study number.

Ethics and Regulatory Considerations

The study was conducted according to Good Clinical Practice, the Declaration of Helsinki and US 21 CFR Part 50—Protection of Human Subjects, and Part 56—Institutional Review Boards. Written, dated informed consent for the study was obtained from all patients before protocol specified procedures are carried out. After signing, each patient was given a copy of their informed consent. Approval of this study was obtained from an Institutional Review Board prior to enrolling patients on study. Consent forms were in a language fully comprehensible to the prospective patient. Consent was documented either by the patient's dated signature or by the signature of an independent witness who records the patient's consent. Each patient's signed informed consent form was kept on file in the clinical research office where individual patients were seen.

Evaluation of Response

(1) Tumor Response Assessment

For patients to be enrolled, there was documented evidence for relapsed or refractory disease (CT scan, investigator description of clinical status of disease, etc). Patients were evaluated by CT scan and physical examination at baseline and every three months thereafter. A second original scan was requested for submission to Favrille for blinded central review. All lymph nodes were evaluated at baseline and followed every 3 months. Response was reported using standard outcome measures for clinical trials (complete response (CR) or complete response/unconfirmed (CRu), partial response (PR), stable disease (SD) and progressive disease (PD)) as defined by Cheson et al. (Cheson B D, et al., “Report of an International Workshop to Standardize Response Criteria for Non-Hodgkin's Lymphomas,” J. Clin Oncol. 17(4):1244 (1999)). Any objective response (CR, CRu, PR) required confirmation at a minimum of 4 weeks later.

(2) Immune Response Evaluation

An immune responder patient was defined as one in whom a humoral and/or cellular immune response to Id-KLH was demonstrated on two or more occasions.

(3) Humoral Immune Response Assessment

Serum for antibody levels specific for idiotype and KLH was obtained at baseline (prior to Rituxan®) and then again prior to each FavId™ dose and at 4 weeks following the last FavId™ treatment. Serum samples were labeled and stored frozen until requested by Favrille and were then tested for reactivity against KLH or the specific vaccinating idiotype by ELISA.

(4) Cellular Immune Response Assessment

The buffy coat from heparinized blood samples was obtained at baseline (prior to Rituxan®) and then again prior to each FavId™ dose and at 4 weeks following the last FavId™ treatment. Samples were labeled and stored in freezing media supplied by Favrille. Cells were tested for capacity to synthesize cytokines or interferon gamma following stimulation with KLH or the specific vaccination idiotype.

Drug Information

Rituxan® was supplied by each study site and administered by intravenous infusion once a week for 4 weeks at a dose of 375 mg/m² according to the manufacturer's instructions.

Toxicities described in the Package Insert for Rituxan include: infusion-related reactions (fever, chills/rigors, hypotension, dyspnea, bronchospasm, angioedema, nausea, vomiting, urticaria, fatigue, headache, pruritus, rhinitis, pain at disease sites); Tumor Lysis Syndrome; hematologic abnormalities (thrombocytopenia, neutropenia, anemia); cardiac arrhythmia; and bullous skin reactions (including toxic epidermal necrolysis).

FavId™ (Id-KLH) was produced from the idiotype gene captured from each patient's lymphoma. This patient specific gene was used to generate idiotype protein using recombinant technology. Resulting purified protein was covalently coupled to keyhole limpet hemocyanin (KLH) prior to patient administration. Formulated FavId™ (Id-KLH) for subcutaneous administration contains 0.5 mg of Id and 0.5 mg of KLH per ml of normal saline; it is supplied in screw-cap plastic vials, and stored prior to administration at −20° C.

FavId™ was administered together with GM-CSF. On the day of FavId™ injection, 250 mcg of GM-CSF was drawn into a plastic tuberculin syringe and added to a thawed vial of FavId™. The vial was gently agitated, and the vial contents drawn up using an 18-gauge needle. After the entire contents have been drawn up, the 18-gauge needle was replaced by a 25-gauge needle for SQ injection. This procedure is important to ensure that all particulates are obtained from the vial. The dose was split equally between the two upper or lower extremities (e.g. right arm and left arm). The sites of injection should be rotated between upper and lower extremities.

(1) Expected Side Effects—FavId™

Id-KLH and Id-KLH with soluble GM-CSF has been previously administered to patients with non-Hodgkin's lymphoma and multiple myeloma (Hsu et al, 1997; Bendandi M, et al., 1999; Massaia, et al., 1999; Harris N L, et al., “World Health Organization Classification of Neoplastic Diseases of the Hematopoietic and Lymphoid Tissues: Report of the Clinical Advisory Committee Meeting—Airlie House, Virginia, November 1997,” 17(12):3835-49 (1999)). Side effects were minimal, the most frequent of which included local reactions at the site of injection. Potential adverse events are listed below:
(1) local skin reactions at the site of injection: erythema, tenderness, induration, urticaria/rash, pruritus;
(2) fever, myalgias/arthralagias, chills/rigors, nausea, fatigue, headache, thrombocytopenia and other cytopenias, hyperglycemia, vomiting, hypotension; and
(3) in addition there may also be a risk of autoimmune disease development, although to date no evidence of this has been seen in any Id immunotherapy study.

(2) GM-CSF (Sargramostim: LEUKINE®)

The GM-CSF used in this study was glycosylated, human granulocyte-macrophage colony stimulating factor (rhu GM-CSF) produced by recombinant DNA technology in a yeast (S. cerevisiae) expression system and manufactured by Immunex Corporation. As supplied LEUKINE® Liquid contains 500 mcg/mL (2.8×10⁶ IU/mL) of sargramostim and was supplied by Favrille, Inc. LEUKINE® liquid was kept refrigerated at 2-8° C. (36-46° F.), and neither frozen nor shaken. Vials were discarded after 20 days.

A flat 250 mcg dose of GM-CSF was administered together with FavId™ split into two injection sites on day 1 of each treatment. The dose of GM-CSF alone (250 mcg) was also split (125 mcg per injection site) and injected SQ on days 2-4, as close as possible to the day 1 FavId™ injection bilateral sites. The administration of GM-CSF alone was not necessarily performed at the principal investigator's site but may have been self-administered by the patient or their designee following appropriate instruction.

Toxicities described in patients receiving GM-CSF may include: fever, chills, diaphoresis, myalgias, fatigue, malaise, headache, dizziness, dyspnea, bronchospasm, pleural effusion, anorexia, indigestion, nausea, vomiting, diarrhea, injection site tenderness, urticaria, rash, pruritus, hypersensitivity reaction, bone pain, thromboembolic events, phlebitis, hypotension, peripheral edema, leukocytosis, thrombocytosis, hepatic enzyme abnormalities and bilirubin elevation.

Occupational Safety

FavId™ is not expected to pose significant occupational safety risks to investigational staff under normal conditions of use and administration. However, precautions should be taken to avoid direct contact with study medication. Rituxan® and Leukine® should be handled according to the package insert.

Adverse Events

(1) Adverse Event

An adverse event (AE) is any untoward medical occurrence in a patient or clinical investigation patient administered a pharmaceutical product, regardless of causality assessment. An adverse event can therefore be any unfavorable and unintended sign (including an abnormal laboratory finding), symptom or disease temporally associated with the use of a medicinal (investigational) product, whether or not considered related to the medicinal (investigational) product.

No further reporting of new adverse events is required after the initiation of any subsequent chemotherapy.

(2) Serious Adverse Events

A serious adverse event (SAE) is any untoward medical occurrence that at any dose (including overdose) is fatal, life threatening, requires or prolongs hospitalization, results in permanent disability, is a congenital anomaly, requires medical/surgical intervention, is a medically significant event.

All SAEs must be reported to Favrille, Inc. within 24 hours of their occurrence. A Serious Adverse Event Form will be supplied by Favrille and completed by the study site. A MedWatch Form will then be generated and, pending review by the Principal Investigator, filed with the FDA.

Patient Completion and Withdrawal

(1) Indication for Taking Patients Off Study

Patients with clear evidence of progressive disease (new lesions; >50% increase in SPD) at the completion of all 6 doses of immunotherapy were removed from the study. Note: Patients with apparent disease progression completed all 6 doses of immunotherapy because of the potential for a late immune response provided the investigator felt that this was in the patient's best interest.

Any patients that experienced grade 3 or 4 toxicity (except rate-of-infusion related reactions or hematologic toxicity) secondary to Rituxan® or a second grade 3 local injection site toxicity from FavId™ and GM-CSF following a reduction in the dose of GM-CSF were removed from the study. Patients were also removed from the study if continued treatment was not in the patient's best interest as judged by the investigator, or if patient requested to be taken off study.

Patients were to be removed from the study if the patient had completed the full course of treatment and final evaluation. Note that patients with an objective response (CR, PR) or lack of progressive disease (PD) following a full course of treatment (i.e., 4 doses of Rituxan® and 6 doses of FavId™) optionally continued to receive FavId™ immunotherapy every 2 months for 6 doses and then every 3 months until documented disease progression.

(2) Study Completion

The study is considered completed when the target enrollment is met and all patients complete a full course of study treatment (4 doses of Rituxan® and 6 doses of FavId™), plus a minimum follow-up period of 1 month.

Data Evaluation

(3) Statistics and Sample Size

For this phase II study, as is normal, patient sample size was not statistically driven. A total of 20 evaluable patients were entered over twelve to eighteen months.

(4) Definition of Evaluable Patients

Patients were only fully evaluated for objective response after receiving all four doses of Rituxan® and all six doses of FavId™ in order to be considered. All patients receiving at least 2 doses of FavId were evaluated for any evidence of immune response. Patients were evaluated for objective responses at two time points: (1) at three months following initiation of Rituxan® dosing, and (2) at the end of all six FavId™ treatment courses. Any objective response was confirmed at least four weeks later. A patient may have been considered to have progressive disease any time during Rituxan® or FavId™ therapy. Any patient who received any study treatment was assessed for safety and toxicity.

(5) Reporting of Outcomes

Descriptive statistics for immune response, safety and efficacy were provided at the conclusion of the study. Objective response rates of CR, PR, SD, and PD were and will be reported for those patients determined to be evaluable. Safety was and will be reported as percent of patients experiencing a given adverse event.

### Example 2

### Treatment of a B-Cell NHL Patient with No Prior Lymphoma Therapy Using a Specific Binding Cytoreductive Agent and Autologous Id Protein

FavId™ is a tumor-specific B-cell recombinant immunoglobulin idiotype (Id) protein which is complexed with KLH and used for the induction of active immunity in patients with indolent NHL.

Objective: This study was conducted to evaluate the ability of FavId™ to increase or prolong the objective response rate following rituximab compared to historical data for rituximab alone from Witzig et al. and to evaluate the ability of patients treated with FavId™ following rituximab to mount an immune response to KLH and idiotype.

Eligibility: Patients with grade 1 or 2 follicular NHL who were treatment naïve.

Treatment: As described supra in Example 1, Rituximab 375 mg/m² was given weekly for 4 weeks. Two months following the last dose of rituximab, FavId™ (1 mg) treatment commenced with FavId™ administered s.q. once a month for 6 months. GM-CSF 250 mcg was given at the FavId™ injection site on Days 1-4. Patients with at least SD after 6 doses of FavId™ were eligible to continue receiving the vaccine every other month for 6 doses and then every 3 months until disease progression. A total of 40 patients were enrolled and receive treatment with rituximab and FavId™.

FavId™ is produced in part according to the methods set forth in PCT Application No. PCT/US01/25204, entitled “Method and Composition for Altering a B Cell Mediated Pathology” published on Feb. 21, 2002 as WO 02/13862.

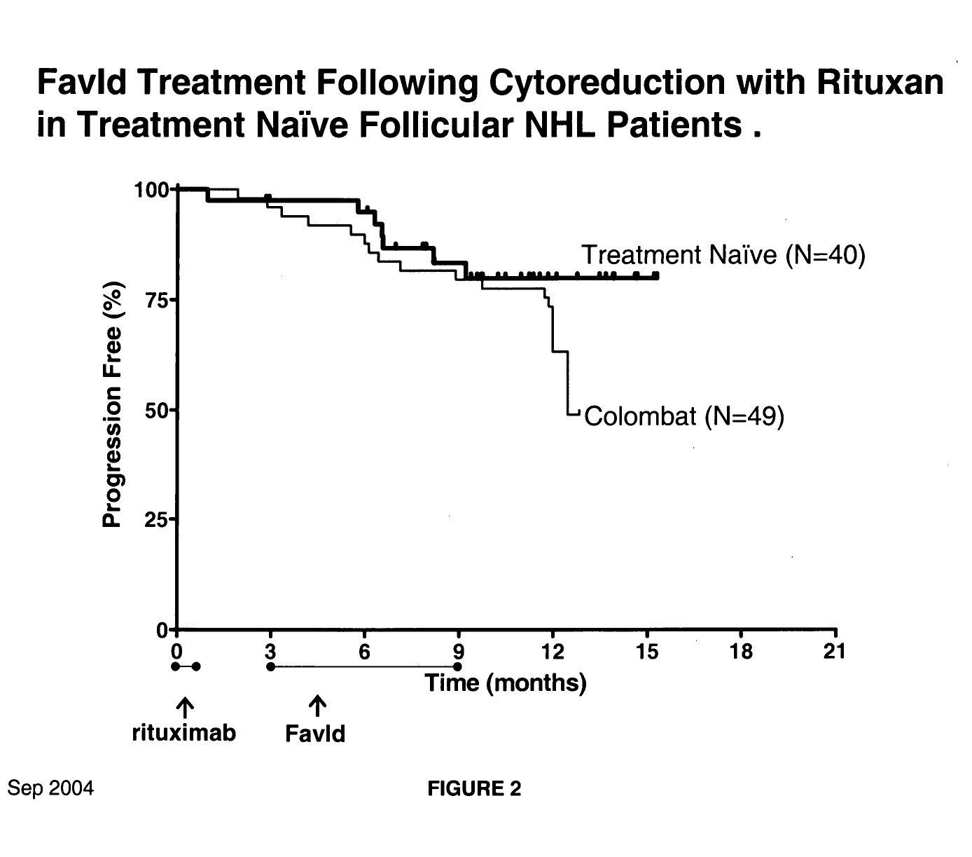

Conclusion: FavId™ administered in this dose and schedule following rituximab is extremely well tolerated and increases the response rate and extend the time to progression following rituximab as seen in FIG. 2. A randomized, double-blind trial of rituximab+FavId™ is being initiated.

### Example 3

### Use of Dendritic Cells as Part of the Combination Therapy

Dendritic cells have been shown to play a critical role in the initiation of immune responses. To take advantage of this, several groups seeking to immunize a patient with cancer antigens have withdrawn dendritic cells from a patient, pulsed them with an immunogen, and re-inoculated them into the patient. Indeed, one group has referred to them as “nature's adjuvants” ((Thurner B, et al., “Vaccination with mage-3A1 peptide-pulsed mature, monocyte-derived dendritic cells expands specific cytotoxic T cells and induces regression of some metastases in advanced stage IV melanoma,” J Exp Med. 190:1669-1678 (1999)) (citing to Schuler G & R M Steinman, “Dendritic cells as adjuvants for immune-mediated resistance to tumors,” J Exp Med., 186(8):1183-87 (1997)).

Among the cancers studied are: B cell lymphomas (Hsu, F J, et al., Vaccination of patients with B-cell lymphoma using autologous antigen-pulsed dendritic cells. Nat Med. 2:52-58 (1996), (Timmerman J M, et al., “Idiotype-pulsed dendritic cell vaccination for B-cell lymphoma: clinical and immune responses in 35 patients,” Blood 99(5):1517-26 (2002)); melanoma (Nestle F O, et al., “Vaccination of melanoma patients with peptide- or tumor lysate-pulsed dendritic cells,” Nat Med. 4:328-332 (1998)), (Thurner B, et al., “Vaccination with mage-3A1 peptide-pulsed mature, monocyte-derived dendritic cells expands specific cytotoxic T cells and induces regression of some metastases in advanced stage IV melanoma,” J Exp Med. 190:1669-1678 (1999)); prostate carcinoma (Murphy, et al., “Phase II prostate cancer vaccine trial: report of a study involving 37 patients with disease recurrence following primary treatment,” Prostate 39:54-59 (1999)), (Murphy G P, et al., “Infusion of dendritic cells pulsed with HLA-A2-specific prostate-specific membrane antigen peptides: a phase II prostate cancer vaccine trial involving patients with hormone-refractory metastatic disease,” Prostate. 38:73-78 (1999)); and renal cell carcinoma (Kugler A, et al., “Regression of human metastatic renal cell carcinoma after vaccination with tumor cell-dendritic cell hybrids,” Nat Med. 6:332-336 (2000)) and carcinoembryonic antigen-expressing cancers (Fong L, et al., “Altered peptide ligand vaccination with F1t3 ligand expanded dendritic cells for tumor immunotherapy,” Proc Natl Acad Sci USA. 98:8809-8814 (2001)).

As can be seen, loading dendritic cells has been used for studies treating several cancers, but none have taken advantage of rapid cloning of an idiotype protein followed by its expression to closely follow therapy with a specific binding cytoreductive agent.

Dendritic cells may be isolated and loaded with Id proteins by the methods of Timmerman et al. Timmerman et al. isolated dendritic cells from leukapheresis products by a series of density gradient centrifugation steps. Peripheral blood mononuclear cells (PBMCs) are obtained by leukapheresis using a COBE cell separator apparatus or similar techniques. This is followed by Ficoll-Hypaque sedimentation (Pharmacia, Uppsala, Sweden). Monocytes are then removed by a discontinuous (50%) Percoll gradient (Pharrnacia). This high-density fraction is cultured overnight in RPMI 1640 plus 10% human autologous serum or 10% AB serum in Teflon vessels (Savillex, Minneapolis, Minn.) along with antigen at 2 μg/mL. High-density lymphocytes are removed using a 15% metrizamide gradient (Sigma, St Louis, Mo.). The low-density, dendritic cell-enriched fraction is recultured overnight in Id protein at an increased concentration of Id protein (50 μg/mL). Following this last incubation, the dendritic cells are harvested, washed, and resuspended in sterile saline for re-infusion (Timmerman J M, et al., “Idiotype-pulsed dendritic cell vaccination for B-cell lymphoma: clinical and immune responses in 35 patients,” Blood 99(5):1517-26 (2002)).

Note that Timmerman based their immunogen concentrations for the sequential overnight incubations based in part on the limited availability of Id proteins using their methods. With the improved methods of generating Id protein in the present invention, this limitation may be removed.

Thurner et al. used a slightly different protocol for the production of loaded dendritic cells. Thus, dendritic cells may also be obtained using a single leukapheresis as described in Thurner et al., 1999 (Thurner, B., et al., “Generation of large numbers of fully mature and stable dendritic cells from leukapheresis products for clinical application,” J. Immunol. Methods 223:1 -15 (1999)). After withdrawing cells from a patient, the PBMCs are isolated by means such as on Lymphoprep™ (Nycomed Pharma) and are divided into three fractions. The first fraction of 10⁹ PBMCs is cultured on bacteriological petri dishes coated with human Ig (100 μg/ml) in complete RPMI 1640 medium (BioWhittaker) supplemented with 20 μg/ml gentamicin, 2 mM glutamine, and 1% heat-inactivated human plasma for 24 hours. This generates monocyte-conditioned medium (MCM) for subsequent use to stimulate DC maturation. The second fraction of PBMCs is used as the source for the dendritic cells to be loaded with Id protein, or portions of the Id protein, and returned to the patient, as well as for any required tests. Such tests could include the delayed-type hypersensitivity (DTH) test I. Adherent monocytes are cultured in 1,000 U/ml GM-CSF (10×10⁷ U/mg) and 800 U/ml IL-4 (purity>98%; 4.1×10⁷ U/mg in a bioassay using proliferation of human IL-4R+CTLL for six days; this is followed by the addition of MCM to drive the maturation of the dendritic cells. MCM may be supplemented in some patients with 10 ng/ml GMP-rhu TNF-α (purity>99%; 5×10⁷) to assure full maturation of DCs. Mature DCs are harvested on day 7. The third fraction of PBMCs is frozen in aliquots and stored in liquid nitrogen for future use (Thurner B, et al., “Vaccination with mage-3A1 peptide-pulsed mature, monocyte-derived dendritic cells expands specific cytotoxic T cells and induces regression of some metastases in advanced stage IV melanoma,” J Exp Med. 190:1669-1678 (1999).

Dendritic cells are pulsed with immunogens, in this case, FavId™, at concentrations from 10 μg/ml to 10 μM. After seven days, the dendritic cells are harvested, resuspended in complete medium, washed, and pulsed again with the peptide at 30 μM for 60 min at 37° C. Dendritic cells are washed and resuspended in PBS for return to the patient (Thurner, et al., 1999).

In preferred embodiments, the loading of dendritic cells is within twelve weeks following treatment with a specific binding cytoreductive agent, such as an anti-CD22 antibody.

### Example 4

### Use of Langerhans Cells as Part of the Combination Therapy

Kumamoto et al., have also recently developed a technique to load Langerhans cells trapped in lymph nodes with ethylene-vinyl-acetate polymer rods. Using this technology, antigen presenting cells are loaded with an immunogen (tumor associated antigen) in vivo (Kumamoto et al., Nat. Biotech. 20:64-69 (2002)). The immunogen used in the present invention is the Id protein expressed in an in vitro system such as bacterial cells, yeast, mammalian cells, or insect cells. In preferred embodiments, the loading of the Langerhans cells is within twelve weeks following treatment with a specific binding cytoreductive agent, such as an anti-CD22 antibody.

### Example 5

### Treatment of a Patient Using a Specific Binding Cytoreductive Agent and Autologous Id Protein Produced in Mammalian Cells

Anti-CD20 antibodies are approved for treatments for patients certain types of B-cell non-Hodgkin's lymphoma. They are used in conjunction with immunizations of Id protein conjugated to KLH plus a cytokine or chemokine such as GM-CSF.

All patients have a lymph node biopsy to obtain tissue for morphological classification, immunophenotypic characterization and to provide material for the in vitro generation of Id protein.

Following characterization of the lymphoma, remaining biopsy cell suspensions are stored in liquid nitrogen for archival purposes. This material may be used for in vitro immunological response studies to either detect the presence of tumor specific antibodies in the sera of treated patients or detect tumor reactive T lymphocytes from the peripheral blood of treated patients.

Eligible patients receive 4 weekly infusions of Rituxan® at standard doses followed within 12 weeks by the first of 6 monthly subcutaneous Id protein conjugated to KLH injections together with GM-CSF. Patients with an objective response (CR or PR) or lack of progressive disease continue to receive Id protein/GM-CSF treatments every other month for 6 treatments and then every 3 months provided there is no evidence of disease progression. Serum anti-idiotypic and anti-KLH antibody are assessed from samples obtained at baseline (prior to anti-CD20 antibody) and before each Id protein treatment and at 4 weeks following the last Id protein treatment. Idiotype-specific and KLH-specific proliferation of PBMC are also measured from samples obtained at baseline (prior to anti-CD20 antibody) and before each Id protein treatment and four weeks following the completion of Id protein therapy.

Administration

(1) The anti-CD20 antibody is administered as an intravenous infusion according to the package insert once a week for 4 weeks or as the treating physician deems appropriate. Prescribing details as supplied by the manufacturer are used.

(2) Id protein/KLH and GM-CSF

Id protein/KLH and GM-CSF treatments are begun approximately 8 weeks following the completion of anti-CD20 antibody treatment, or as soon as permitted by the completion of the production of the Id protein. Id protein, 1 mg (0.5 mg of tumor Id protein conjugated to 0.5 mg of KLH carrier protein) and 250 mcg of soluble GM-CSF are generally used on prior clinical experience with similar treatment schedules and doses.

Patients receive a subcutaneous injection of Id protein with GM-CSF (split into two injection sites) once a month for six months. A flat dose of 250 mcg of GM-CSF alone is administered by subcutaneous injection for 3 days following each Id protein injection. All GM-CSF injections are also split (125 mcg per injection site) and given in close proximity to the FavId™ injection sites, as close to the exact sites of the FavId™ Day 1 injection sites as possible. The injection sites are rotated between the upper and lower extremities each month.

Drug Information

The anti-CD20 antibody is obtained from the manufacturer and used according to the recommended procedures. Id protein is produced from the idiotype gene captured from each patient's lymphoma. This patient specific gene is used to generate a gene expressing the idiotype region inserted into a vector encoding immunoglobulin light chains and heavy chains using recombinant technology.

The recombinant gene is expressed in mammalian cells using standard techniques known in the art. For example, the expression vector encoding the Id protein may be transformed into CHO cells together with the DHFR gene, followed by amplification, as described in U.S. Pat. Nos. 4,399,216, 4,634,665, and 5,179,017 (Axel patents).

Resulting purified protein is covalently coupled to keyhole limpet hemocyanin (KLH) prior to patient administration. Formulated FavId™ (Id-KLH) for subcutaneous administration contains 0.5 mg of Id and 0.5 mg of KLH per ml of normal saline; it is supplied in screw-cap plastic vials, and stored prior to administration at −20° C. FavId™ is administered together with GM-CSF as described elsewhere in the specification.

### Example 6

### Treatment of a Patient Using a Specific Binding Cytoreductive Agent and Autologous Id Protein Produced in Yeast Cells

Anti-CD20 antibodies are approved for treatments for patients certain types of B-cell non-Hodgkin's lymphoma. They are used in conjunction with immunizations of Id protein conjugated to KLH plus a cytokine or chemokine such as GM-CSF.

All patients have a lymph node biopsy to obtain tissue for morphological classification, immunophenotypic characterization and to provide material for the in vitro generation of Id protein.

Following characterization of the lymphoma, remaining biopsy cell suspensions are stored in liquid nitrogen for archival purposes. This material may be used for in vitro immunological response studies to either detect the presence of tumor specific antibodies in the sera of treated patients or detect tumor reactive T lymphocytes from the peripheral blood of treated patients.

Eligible patients receive 4 weekly infusions of Rituxan® at standard doses followed within 12 weeks by the first of 6 monthly subcutaneous Id protein conjugated to KLH injections together with GM-CSF. Patients with an objective response (CR or PR) or lack of progressive disease may continue to receive Id protein/GM-CSF treatments every other month for 6 treatments and then every 3 months provided there is no evidence of disease progression. Serum anti-idiotypic and anti-KLH antibody are assessed from samples obtained at baseline (prior to anti-CD20 antibody) and before each Id protein treatment and at 4 weeks following the last Id protein treatment. Idiotype-specific and KLH-specific proliferation of PBMC are also measured from samples obtained at baseline (prior to anti-CD20 antibody) and before each Id protein treatment and four weeks following the completion of Id protein therapy.

Administration

(1) The anti-CD20 antibody is administered as an intravenous infusion according to the package insert once a week for 4 weeks or as the treating physician deems appropriate. Prescribing details as supplied by the manufacturer are used.

(2) Id protein/KLH and GM-CSF

Id protein/KLH and GM-CSF are begun approximately 8 weeks following the completion of anti-CD20 antibody treatment, or as soon as permitted by the completion of the production of the Id protein. Id protein, 1 mg (0.5 mg of tumor Id protein conjugated to 0.5 mg of KLH carrier protein) and 250 mcg of soluble GM-CSF are generally used on prior clinical experience with similar treatment schedules and doses.

Patients receive a subcutaneous injection of Id protein with GM-CSF (split into two injection sites) once a month for six months. A flat dose of 250 mcg of GM-CSF alone is administered by subcutaneous injection for 3 days following each Id protein injection. All GM-CSF injections are also split (125 mcg per injection site) and given in close proximity to the FavId™ injection sites, as close to the exact sites of the FavId™ Day 1 injection sites as possible. The injection sites are rotated between the upper and lower extremities each month.

Drug Information

The anti-CD20 antibody is obtained from the manufacturer and used according to the recommended procedures. Id protein is produced from the idiotype gene captured from each patient's lymphoma. This patient specific gene is used to generate a gene expressing the idiotype region inserted into a vector encoding immunoglobulin light chains and heavy chains using recombinant technology.

The recombinant gene is expressed in yeast cells using standard techniques known in the art. For example, the expression vector encoding the Id protein may be transformed into yeast cells and expressed as described in U.S. Pat. Nos. 4,546,082, 5,162,498, or 6,183,989.

Resulting purified protein is covalently coupled to keyhole limpet hemocyanin (KLH) prior to patient administration. Formulated Favld™ (Id-KLH) for subcutaneous administration contains 0.5 mg of Id and 0.5 mg of KLH per ml of normal saline; it is supplied in screw-cap plastic vials, and stored prior to administration at −20° C. FavId™ is administered together with GM-CSF as described elsewhere in the specification.

### Example 7

### Treatment of a Patient Using a Specific Binding Cytoreductive Agent and Autologous Id Protein Produced in Bacteria

Anti-CD20 antibodies are approved for treatments for patients certain types of B-cell non-Hodgkin's lymphoma. They are used in conjunction with immunizations of Id protein conjugated to KLH plus a cytokine or chemokine such as GM-CSF.

All patients have a lymph node biopsy to obtain tissue for morphological classification, immunophenotypic characterization and to provide material for the in vitro generation of Id protein.

Following characterization of the lymphoma, remaining biopsy cell suspensions are stored in liquid nitrogen for archival purposes. This material may be used for in vitro immunological response studies to either detect the presence of tumor specific antibodies in the sera of treated patients or detect tumor reactive T lymphocytes from the peripheral blood of treated patients.

Eligible patients receive 4 weekly infusions of Rituxan® at standard doses followed within 12 weeks by the first of 6 monthly subcutaneous Id protein conjugated to KLH injections together with GM-CSF. Patients with an objective response (CR or PR) or lack of progressive disease may continue to receive Id protein/GM-CSF treatments every other month for 6 treatments and then every 3 months provided there is no evidence of disease progression. Serum anti-idiotypic and anti-KLH antibody are assessed from samples obtained at baseline (prior to anti-CD20 antibody) and before each Id protein treatment and at 4 weeks following the last Id protein treatment. Idiotype-specific and KLH-specific proliferation of PBMC are also measured from samples obtained at baseline (prior to anti-CD20 antibody) and before each Id protein treatment and four weeks following the completion of Id protein therapy.

Administration

(1) The anti-CD20 antibody is administered as an intravenous infusion according to the package insert once a week for 4 weeks or as the treating physician deems appropriate. Prescribing details as supplied by the manufacturer are used.

(2) Id protein/KLH and GM-CSF

Id protein/KLH and GM-CSF are begun approximately 8 weeks following the completion of anti-CD20 antibody treatment, or as soon as permitted by the completion of the production of the Id protein. Id protein, 1 mg (0.5 mg of tumor Id protein conjugated to 0.5 mg of KLH carrier protein) and 250 mcg of soluble GM-CSF are generally used on prior clinical experience with similar treatment schedules and doses.

Patients receive a subcutaneous injection of Id protein with GM-CSF (split into two injection sites) once a month for six months. A flat dose of 250 mcg of GM-CSF alone is administered by subcutaneous injection for 3 days following each Id protein injection. All GM-CSF injections are also split (125 mcg per injection site) and given in close proximity to the FavId™ injection sites, as close to the exact sites of the FavId™ Day 1 injection sites as possible. The injection sites are rotated between the upper and lower extremities each month.

Drug Information

The anti-CD20 antibody is obtained from the manufacturer and used according to the recommended procedures. Id protein is produced from the idiotype gene captured from each patient's lymphoma. This patient specific gene is used to generate a gene expressing the idiotype region inserted into a vector encoding immunoglobulin light chains and heavy chains using recombinant technology.

The recombinant gene is expressed in bacterial cells using standard techniques known in the art. For example, the expression vector encoding the Id protein may be transformed into bacterial cells and expressed as described in U.S. Pat. Nos. 4,816,567, and 6,331,415 (Cabilly patents).

Resulting purified protein is covalently coupled to keyhole limpet hemocyanin (KLH) prior to patient administration. Formulated FavId™ (Id-KLH) for subcutaneous administration contains 0.5 mg of Id and 0.5 mg of KLH per ml of normal saline; it is supplied in screw-cap plastic vials, and stored prior to administration at −20° C. FavId™ is administered together with GM-CSF as described elsewhere in the specification.

### Example 8

### Treatment of a Patient Using a Specific Binding Cytoreductive Agent and Autologous Id Protein During Treatment with a Cytoreductive Agent

Maintenance Rituximab plus FavId™ and GM-CSF Immunotherapy in Patients with Indolent B-Cell Lymphoma

The aim of this example is the treatment of indolent non-Hodgkin's lymphoma (follicular small cleaved cell, follicular mixed, small lymphocytic [SLL]). The example is planned to evaluate the safety of the combination of maintenance rituximab and Favld in treatment naïve patients (ECOG status 0, 1 or 2) based on event free survival and response rate. The example is further intended to evaluate the development of an anti-Id immune response (both cellular and humoral following Favld.

Patients will initially begin rituximab treatment at a standard dose and schedule (e.g., 375 mg/m²/week×4). As soon as FavId™ becomes available (approximately twelve weeks after beginning treatment), patients will begin receiving monthly subcutaneous injections of FavId™. Thus the administration of the specific-binding cytoreductive agent and the autologous Id protein continue in an overlapping, or parallel, manner. GM-CSF will be administered together with FavId™ and alone on the second, third and fourth days following administration of FavId™. Treatment with rituximab will be repeated every six months for four total cycles with FavId™/GM-CSF not being given on those months when rituximab is administered. Starting on month 12, the administration frequency for FavId™/GM-CSF will decrease to every two months. If patients show no disease progression after twenty-five months, they may continue to receive FavId™/GM-CSF every three months until disease progression.

Patients are evaluated according to clinical laboratory data, physical exams, observations for adverse events. Outcomes are measured by EFS (Event Free Survival), response rate at month six, overall response rate (Response Rate), TTP, DR, and immune response to FavId™ (both cellular and humoral). Efficacy data are analyzed by intent to treat (ITT) and by an efficacy evaluable (EE) patient populations.

FavId™ is generally administered at a concentration of 1 mg/ml in solution. GM-CSF is administered at 500 mcg/ml. Rituximab is generally administered at 10 mg/ml. The minimal toxicity of FavId™ and rituximab should allow for the combination of both agents at full dose and schedule. Without being bound by any theory, the B cell depletion induced by rituximab may lead to an increased cell mediated immune response to subsequent FavId™ immunization.

The schedule of maintenance rituximab of Hainsworth et al. (Hainsworth J D, et al., J Clin Oncol 20(20):4261 4267 (2002)) is used along with the schedule of FavId™ used in prior FavId™ studies (initial monthly injections slowing to every two month injections after the first 6 injections, and then every 3 month injections after the first one to two years). By administering rituximab and FavId™ on different days, it is easier to assign side effects to a specific drug rather than to the entire treatment regimen.

Study Population

To be involved in the study, patients must satisfy the following criteria: (1) ≧18 years of age; (2) ECOG Performance status of 0, 1 or 2; (3) No prior anticancer therapy except for local irradiation therapy; (4) Tumor accessible for biopsy, or available biopsy material judged by Favrille to be suitable for preparation of FavId™; (5) Measurable or evaluable disease following node biopsy; (6) Histologically confirmed Grade 1 or 2 follicular B cell lymphoma or small lymphocytic lymphoma (SLL) by the WHO classification system; (7) Judged suitable for treatment with rituximab by the treating physician; (8) WBC count ≧3,000/mm3; (9) Platelets >100,000/mm3; (10) Total Bilirubin ≦2 mg/dL; (11) AST and ALT ≦2×Upper Limit of Normal; (12) Creatinine ≦1.5 mg/dL; (13) Patients of childbearing potential and patients who have partners of childbearing potential must agree to use contraception from the signing of the informed consent until 30 days after they discontinue the study; and (14) Ability to understand the nature of the study and voluntarily give informed consent.

Treatment Plan

Patients will undergo FNA of their lymphoma to obtain biopsy for production of Favld. Patients initially receive rituximab at 375 mg/m² IV, once per week for 4 consecutive weeks.

Patients are reevaluated for response to rituximab at week 9 (month 3). Patients with objective response (CR, CRᵤ, PR) or stable disease proceed with maintenance therapy. Patients with'progression of lymphoma are removed from study.

All patients who begin maintenance therapy may receive repeat courses of rituximab at 6-month intervals and intermittent injections of FavId/GM-CSF for a total of 24 months.

During the first year of maintenance therapy, FavId/GM-CSF is administered monthly, beginning month 4 (week 13). A FavId/GM-CSF combination is administered concurrently with rituximab, during the first 12 months, FavId injections are generally administered at months 4, 5, 6, 8, 9, 10, and 11. During the second year, FavId/GM-CSF is administered every other month (except during months when rituximab is administered). Therefore, injections during the second year are at months 14, 16, 18, 20, 22, and 24. For patients with continuing response after completing 2 years of therapy, FavId/GM-CSF is continued at 3-month intervals until lymphoma progression.

On days FavId/GM-CSF is administered, Favld protein (0.5 mg) and KLH protein (0.5 mg) is mixed with GM-CSF 250 μg and injected subcutaneously. On days 2-4, GM-CSF 250 μg SQ will be administered and is injected near the site of the Favld injection. The doses on days 2-4 may be self administered at home by the patient.

Rituximab Maintenance:

Maintenance courses of rituximab are given every 6 months for 3 maintenance courses (i.e. beginning month 7, 13, and 19). Each maintenance course of rituximab generally consists of 4 weekly doses, (375 mg/m² IV infusion).

Pretreatment Evaluation:

The studies that are necessary for pretreatment evaluation, and during the first 12 months of treatment, are summarized in Table 1. All patients have a biopsy for production of Favld antibody. Radiologic evaluations are performed within 1 month of study entry. Laboratory tests are performed within 1 week of study entry. Patients will have bone marrow aspiration and biopsy, if these studies have not been performed within 6 months of study entry.

TABLE 1
Pretreatment Studies and Evaluations During the First 12 Months

| | | Month | | | | | | | | | | |
|---|---|---|---|---|---|---|---|---|---|---|---|---|
| | Pretreatment | 2 | 3 | 4 | 5 | 6 | 7 | 8 | 9 | 10 | 11 | 12 |
| History Physical | X | X | X | X | X | X | X | X | X | X | X | X |
| Weight, PS | X | | | | | | | | | | | |
| Tumor Measurements | X | | X | | | | | | | | | |
| CBC, platelets, diff | X | X | X | X | X | X | X | X | X | X | X | X |
| CMP + LDH + uric acid | X | X | X | X | X | X | X | X | X | X | X | X |
| HIV serology | X | | | | | | | | | | | |
| Pregnancy test | Xa | | | | | | | | | | | |
| CT chest/abdomen | X | | X | | | | X | | | X | | |
| CT head | Xb | | | | | | | | | | | |
| Bone marrow aspiration bx | Xc | | Xf | | | | | | | | | |
| Biopsy of lymphoma | Xd | | | | | | | | | | | |
| Humoral/cellular immunity | Xe | | | X | X | X | | X | X | X | X | |
| aOnly in women of childbearing potential | | | | | | | | | | | | |
| bOnly if clinically indicated | | | | | | | | | | | | |
| cFlow cytometry for detection of bcl-2 gene rearrangements should be included. Not required in patients who have had bone marrow aspiration within 6 months. | | | | | | | | | | | | |
| dRequired for specimen to make FavId antibody (see Appendix I). | | | | | | | | | | | | |
| eBaseline and prior to each dose of FavId. Specimens will be stored and sent to Favrille (Appendix III). | | | | | | | | | | | | |
| fOnly if initially abnormal, and necessary to confirm CR (include flow cytometry) Patients are reevaluated for treatment response at 3-month intervals. Patients are examined monthly during the first year, and monthly CBC, platelets, differential, and chemistry are performed. Tumor restaging and tumor measurements continue at 6-month intervals. CBC, platelets, CMP, and tests for humoral/cellular immunity are performed | | | | | | | | | | | | |
| # prior to each scheduled dose of FavId/GM-CSF, and prior to each course of rituximab. | | | | | | | | | | | | |

TABLE 2
Patient Evaluation During Months 12-25

| | Month | | | | | | | | | | | | |
|---|---|---|---|---|---|---|---|---|---|---|---|---|---|
| | 13 | 14 | 15 | 16 | 17 | 18 | 19 | 20 | 21 | 22 | 23 | 24 | 25 |
| History/physical | X | | | X | | X | X | | | X | | | X |
| Weight/PS | X | | | X | | X | X | | | X | | | X |
| Tumor measurements | X | | | X | | | X | | | X | | | X |
| CBC, platelets, diff | X | X | | X | | X | X | X | | X | | X | X |
| CMP, LDH, uric acid | X | X | | X | | X | X | X | | X | | X | X |
| CT chest/abdomen | X | | | | | | X | | | | | | X |
| Humoral/cellular immunity | | X | | X | | X | | X | | X | | X | |

At the completion of 2 years of maintenance (month 25), patients are restaged with reassessment of tumor status. If in clinical complete remission, patients have repeat bone marrow aspiration/biopsy at this time, if bone marrow was previously involved, and will confirm a complete remission.

After completion of 2 years maintenance therapy, patients are seen every 3 months with history and physical examination, CBC, differential, chemistry profile, and tumor measurements of any lesions assessable by physical examination. At 6-month intervals, CT scans of the chest and abdomen are repeated, to assess tumor status. These evaluations should continue until the time of documented tumor progression.

Assessment of Efficacy

The following describes the paramters used to determine efficacy.

Tumor Response Assessment.

Responses are defined using the International Workshop Response Criteria: For the complete list of the criteria, see Appendix II summarized in the table below.

| Response | Physical | Lymph | Lymph Node | |
|---|---|---|---|---|
| Category | Examination | Nodes | Masses | Bone Marrow |
| CR | Normal | Normal | Normal | Normal |
| CRᵤ | Normal | Normal | Normal | Indeterminate |
| | Normal | Normal | <75% | Normal or |
| | | | decrease | indeterminate |
| PR | Normal | Normal | Normal | Positive |
| | Normal | ≧50% | ≧50% | Irrelevant |
| | | decrease | decrease | |
| | Decrease in | ≧50% | ≧50% | Irrelevant |
| | liver/spleen | decrease | decrease | |
| Relapse/ | Enlarging | New or | New or | Reappearance |
| progression | liver/spleen; | increased | increased | |
| | new sites | | | |

Adapted from Cheson et al, 1999.

Immune Response Evaluation:

Humoral and cellular immune response analysis is performed on blood samples obtained from each patient using standard techniques.

The following section lists specific information on the drugs used for treatment.

Drug Information

1. FavId™

Description:

FavId™ is manufactured for each patient using genetic information taken from the lymph node biopsy. FavId™ consists of a unique Id protein derived from a patient covalently coupled to keyhole limpet hemocyanin (KLH).

Formulation:

FavId™ for subcutaneous (SQ) administration contains 0.5 mg of Id protein and 0.5 mg of KLH protein per ml of normal saline.

Preparation:

On the day of FavId™ injection, 250 mcg of commercially available GM-CSF is drawn into a plastic tuberculin syringe and set aside. The entire contents of a thawed vial of FavId™ are drawn up into a 3 ml syringe with an 18-gauge needle. Any particulates should be broken up with the needle and care should be taken to ensure that all particulates in the vial are taken up in the syringe intended for patient administration. The plunger should be pulled back to extract any study material from the needle hub and to leave space for the addition of the GM-CSF. After the entire vial contents have been drawn up, the 18-gauge needle should be removed from the 3 ml syringe. The predrawn GM-CSF dose is added to the 3 ml syringe leaving room for air in the syringe barrel. The 18-gauge needle is reattached and the syringe is gently rocked to mix the solutions. The air is expelled and the study solution is split into two tuberculin syringes for administration. 25-gauge needles should be used for administration.

Storage and Stability:

FavId™ is stored at −20° C.

Administration:

On the day of administration, the total FavId™ dose is split equally between either the two upper or two lower extremities (e.g. right arm and left arm). Injection sites are rotated between upper and lower extremities with subsequent administrations.

Expected Side Effects:

Id-KLH (similar to FavId™) both with and without GM-CSF has been previously administered to patients. Side effects were minimal, the most frequent of which included local reactions at the site of injection. Potential adverse events include:
Local skin reactions at the site of injection such as erythema, tenderness, induration, urticaria/rash, pruritus.
Systemic reactions such as fever, myalgias/arthralagias, chills/rigors, nausea, fatigue, headache, thrombocytopenia and other cytopenias, hyperglycemia, vomiting, hypotension.

In addition, there is a theoretical risk of the development of autoimmune disease, although clinical autoimmune disease has yet to be described in a patient receiving and Id-KLH vaccination.

2. GM-CSF (Sargramostim; Leukine®)

Description:

GM-CSF (LEUKINE®) is glycosylated, human granulocyte-macrophage colony stimulating factor (rhu GM-CSF) produced by recombinant DNA technology in a yeast (S. cerevisiae) expression system manufactured by Immunex Corporation. GM-CSF is used according the manufacturer's description and protocol.

3. Rituximab or Other Cytoreductive Agent

Description:

Rituximab or any other cytoreductive agent is used according the manufacturer's description and protocol.

Endpoints (Primary and Secondary):

The following is a description of the endpoints used for the trial.

Primary:

Event Free Survival (EFS) is defined as time from the first dose of rituximab to the time of any of the following events: death, disease progression, or institution of additional (non-protocol specified) treatment.

Secondary Efficacy Endpoints:

Objective Response Rate at Month 6 (Oust prior to second course of rituximab) Objective response rate at 6 months is defined as the percentage of patients who have achieved or maintained either a complete response (CR) or a partial response (PR) at the 6-month evaluation.

Overall Objective Response Rate: Overall objective response rate is defined as the percentage of patients who achieved either a complete response (CR) or a partial response (PR) at any time during study participation.

Time to Progression: Time to progression (TTP) is defined as the time from the first dose of rituximab until the time of the first documentation of progressive disease. Patients who have not progressed, are lost to follow-up, or die prior to progressing will be censored at the time of their last evaluation.

Duration of Response: Duration of response (DR) is defined as the time from the date of the first documentation of response (CR or PR) until the time of the first documentation of progressive disease. Patients who have not progressed, are lost to follow-up, or die prior to progressing will be censored at the time of their last evaluation.

Immune Response: Patients will be considered to be immune response positive if they develop a positive cellular and/or humoral response to their Id protein at any time following the initiation of Id vaccination. Patients will also be evaluated for a positive cellular or humoral immune response to KLH.

Study Populations

Intent-To-Treat Population: The intent-to-treat population (ITT) includes all patients who have received at least one dose of rituximab.

Efficacy Evaluable Population: The efficacy evaluable population (EE) includes all patients who receive rituximab therapy, receive at least one dose of FavId™/GM-CSF, and receive at least one follow-up tumor assessment.

Evaluable For Safety Population: All patients who receive any study treatment (either rituximab or FavId™/GM-CSF) will be evaluable for safety and toxicity.

The invention illustratively described herein can suitably be practiced in the absence of any element or elements, limitation or limitations that is not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalent of the invention shown or portion thereof, but it is recognized that various modifications are possible within the scope of the invention claimed. Thus, it should be understood that although the present invention has been specifically disclosed by preferred embodiments and optional features, modifications and variations of the inventions embodied herein disclosed can be readily made by those skilled in the art, and that such modifications and variations are considered to be within the scope of the inventions disclosed herein. The inventions have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless or whether or not the material to be removed was specifically recited. In addition, where features or aspects of an invention are described in terms of the Markush group, those schooled in the art will recognize that the invention is also thereby described in terms of any individual member or subgroup of members of the Markush group. Further, when a reference to an aspect of the invention lists a range of individual members, as for example, ‘SEQ ID NO:1 to SEQ ID NO:100, inclusive,’ it is intended to be equivalent to listing every member of the list individually, and additionally it should be understood that every individual member may be excluded or included in the claim individually.

The steps depicted and/or used in methods herein may be performed in a different order than as depicted and/or stated. The steps are merely exemplary of the order these steps may occur. The steps may occur in any order that is desired such that it still performs the goals of the claimed invention.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims.

All U.S. patents and applications; foreign patents and applications; scientific articles; books; and publications mentioned herein are hereby incorporated by reference in their entirety as if each individual patent or publication was specifically and individually indicated to be incorporated by reference, including any drawings, figures and tables, as though set forth in full.

1. A method for treating a B cell malignancy in a patient in need of such treatment with a combination therapy comprising contemporaneously co-administering (1) a specific-binding cytoreductive agent and (2) an immunotherapeutic composition comprising an autologous Id protein.

2. A method for treating a B cell malignancy in a patient in need of such treatment with a combination therapy comprising contemporaneously co-administering (1) a specific-binding cytoreductive agent and (2) an immunotherapeutic composition comprising dendritic cells comprising at least part of an autologous Id protein.

3. A method for improving a treatment for a B cell malignancy wherein said treatment for a B cell malignancy comprises administrating a specific-binding cytoreductive agent and wherein said improved treatment comprises contemporaneously co-administering an autologous Id protein.

4. The method of claim 1, 2, or 3 wherein said B cell malignancy has not been previously treated.

5. The method of claim 1, 2, or 3 wherein said B cell malignancy has not been previously treated with chemotherapeutic agents.

6. The method of claim 1, 2, or 3 wherein said B cell malignancy is a relapsed B cell malignancy.

7. The method of claim 1, 2, or 3 wherein said contemporaneously co-administered treatment with said autologous Id protein begins within three months of the last administration of said specific-binding cytoreductive agent.

8. The method of claim 1, 2, or 3 wherein said contemporaneously co-administered treatment with said autologous Id protein begins within two months of the last administration of said specific-binding cytoreductive agent.

9. The method of claim 1, 2, or 3 wherein said contemporaneously co-administered treatment with said autologous Id protein begins within one month of the last administration of said specific-binding cytoreductive agent.

10. The method of claim 1, 2, or 3 wherein said contemporaneously co-administered treatment with said autologous Id protein begins within one week of the last administration of said specific-binding cytoreductive agent.

11. The method of claim 1, 2, or 3 wherein said B cell malignancy is selected from the group consisting of B cell lymphomas and B cell leukemias.

12. The method of claim 11 wherein said B cell lymphoma is selected from the group consisting of Hodgkin's Disease and Non-Hodgkin's Lymphoma.

13. The method of claim 12 wherein said Non-Hodgkin's Lymphoma is low grade, intermediate grade or high grade.

14. The method of claim 12 wherein said Non-Hodgkin's Lymphoma is selected from the group of subtypes consisting of small lymphocytic, follicular and predominantly small cleaved cell, follicular and mixed small cleaved and large cell type, follicular and predominantly large cell type, diffuse small cleaved cell, diffuse mixed small and large cell, diffuse large cell, large cell immunoblastic, lymphoblastic, small non-cleaved Burkitt's and non-Burkitt's type, AIDS-related lymphomas, angioimmunoblastic lymphadenopathy, and monocytoid B-cell lymphoma.

15. The method of claim 12 wherein said Non-Hodgkin's Lymphoma is mantle cell lymphoma.

16. The method of claim 11 wherein said B-cell leukemia is a chronic B-cell leukemia, acute lymphoblastic leukemia of a B-cell lineage, or chronic lymphocytic leukemia of a B-cell lineage.

17. The method of claim 1, 2, or 3 wherein said specific-binding cytoreductive agent is an antigen-binding cytoreductive agent.

18. The method of claim 1, 2, or 3 wherein said specific-binding cytoreductive agent is selected from the group consisting of an anti-CD20 antibody, an anti-CD22 antibody, an anti-B7 antibody, and an anti-CD156 antibody.

19. The method of claim 1, 2, or 3 wherein said autologous Id protein is produced recombinantly.

20. The method of claim 1, 2, or 3 wherein said autologous Id protein is produced recombinantly in insect cell lines.

21. The method of claim 1, 2, or 3 wherein said autologous Id protein is coupled to KLH.

22. The method of claim 1, 2, or 3 wherein said autologous Id protein is encoded by an open reading frame wherein a portion of the DNA sequence of said open reading is obtained from nucleic acid sequence derived from said B cell malignancy from said patient to be treated with said autologous Id protein.

23. The method of claim 1, 2, or 3 wherein said autologous Id protein does not comprise either a full length heavy antibody chain or a full length light antibody chain.

24. The method of claim 1, 2, or 3 wherein said autologous Id protein does not comprise a full length antibody heavy chain.

25. The method of claim 1, 2, or 3 wherein said autologous Id protein does not comprise a fall length antibody light chain.

26. The method of claim 2 wherein said dendritic cells are autologous dendritic cells.

27. The method of claim 1, 2, or 3 wherein said autologous Id protein is produced recombinantly in yeast cells.

28. The method of claim 1, 2, or 3 wherein said autologous Id protein is produced recombinantly in bacterial cells.

29. The method of claim 1, 2, or 3 wherein said autologous Id protein is produced recombinantly in mammalian cells.

30. The method of claim 1, 2, or 3 wherein said autologous Id protein is produced recombinantly in mammalian T lymphoid cells.

31. The method of claim 1, 2, or 3 wherein said contemporaneously co-administered treatment with said autologous Id protein begins before the end of administration of said specific-binding cytoreductive agent and wherein the treatment using said autologous Id protein and said cytoreductive agent continues in parallel.
